(19) United States
(12) Patent Application Publication
(10) Pub. No.: US 2006/0122279 A1
(43) Pub. Date: Jun. 8, 2006

(54) Hydrophobic polyamine amides as potent lipopolysaccharide sequestrants

(75) Inventors: Mark R. Burns, Kenmore, WA (US); Sunil A. David, Lawrence, KS (US)

Correspondence Address: CONNOLLY BOVE LODGE & HUTZ LLP, SUITE 800, 1990 M STREET NW, WASHINGTON, DC, 20036-3425, US

(73) Assignees: MediQuest Therapeutics, Inc., Bothell, WA (US); The University of Kansas, Lawrence, KS (US)

(21) Appl. No.: 11/271,743

(22) Filed: Nov. 14, 2005

Related U.S. Application Data
(60) Provisional application No. 60/627,082, filed on Nov. 12, 2004.

(51) Int. Cl. A61K 31/16

(52) U.S. Cl. 514/616; 514/626

(57) ABSTRACT

Lysine-spermine conjugates with a long-chain aliphatic (C₁₂-C₂₀) substituent at R₁ bind and neutralize bacterial lipopolysaccharides. These compounds reduce lethality in a murine model of lipopolysaccharide-induced shock, and may serve as novel leads for developing novel anti-lipopolysaccharide agents for the therapy of Gram-negative sepsis. These compounds are represented by the formula: 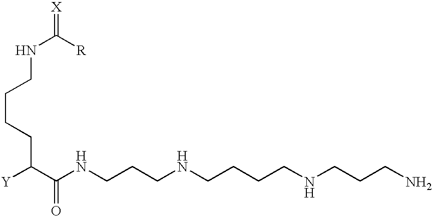
wherein X is O or H, H; R is a hydrophobic C₁₂-C₂₀ chain and Y is -NH₂ or -H; and pharmaceutically acceptable salts thereof and prodrugs thereof.

### CROSS-REFERENCE TO RELATED APPLICATIONS

The present application claims the benefit of U.S. provisional application Ser. No. 60/627,082, filed Nov. 12, 2004, entitled Hydrophobic Polyamine Amides as Potent Lipopolysaccharide Sequestrants, the entire disclosure of which is incorporated herein by reference.

### STATEMENT REGARDING FEDERALLY SPONSORED RESEARCH OR DEVELOPMENT

This work was supported by NIH 1U01 AI054785 (SD) and the US Government may have certain rights in this invention.

### TECHNICAL FIELD

Lipopolysaccharides (LPS), otherwise termed ‘endotoxins’, are outer-membrane constituents of Gram-negative bacteria. Lipopolysaccharides play a key role in the pathogenesis of ‘Septic Shock’, a major cause of mortality in the critically ill patient. Therapeutic options aimed at limiting downstream systemic inflammatory processes by targeting lipopolysaccharide do not exist at the present time. The present inventors have defined the pharmacophore necessary for small molecules to specifically bind and neutralize LPS and, using animal models of sepsis, have shown that the sequestration of circulatory LPS by small molecules is a therapeutically viable strategy. The interactions of a focused library of lysine-spermine conjugates with lipopolysaccharide (LPS) have been characterized. Certain polyamine amides such as lysine-spermine conjugates with the i-amino terminus of the lysinyl moiety derivatized with long-chain aliphatic hydrophobic substituents in acyl or alkyl linkage bind and neutralize bacterial lipopolysaccharides, and along with test results suggest their suitability for the prevention or treatment of endotoxic shock states or sepsis.

### BACKGROUND ART

Endotoxins, or lipopolysaccharides (LPS), the predominant structural component of the outer membrane of Gram-negative bacteria, ^{1;2}play a pivotal role in septic shock, a syndrome of systemic toxicity which occurs frequently when the body's defense mechanisms are compromised or overwhelmed, or as a consequence of antibiotic chemotherapy of serious systemic infections (Gram-negative sepsis).³⁻⁵ Referred to as “blood poisoning” in lay terminology, Gram-negative sepsis is the thirteenth leading cause of overall mortality⁶ and the number one cause of deaths in the intensive care unit,⁷ accounting for more than 200,000 fatalities in the US annually.⁸ Despite tremendous strides in antimicrobial chemotherapy, the incidence of sepsis has risen almost three-fold from 1979 through 2000⁹ and sepsis-associated mortality has essentially remained unchanged at about 45%, both calling to attention the fact that aggressive antimicrobial therapy alone is insufficient in preventing mortality in patients with serious illnesses, and emphasizing an urgent, unmet need to develop therapeutic options specifically targeting the pathophysiology of sepsis.

The presence of LPS causes a widespread activation of the innate immune response,^{10;11} leading to the uncontrolled production of numerous inflammatory mediators, including tumor necrosis factor-α(TNF-α), interleukin-1 β (IL-1β), and interleukin-6 (IL-6), primarily by cells of the monocyte/macrophage lineage.^{12;13} The unregulated overproduction of these mediators, as well as others, such as nitric oxide produced by the endothelial cell,^{14;15} leads to a systemic inflammatory response characterized by fever, hypotension, coagulopathy, hemodynamic derangement, tissue hypoperfusion, and multiple organ failure,^{16;17} culminating frequently in death.

The therapy of septic shock remains primarily supportive, and specific modalities aimed at limiting the underlying pathophysiology are, unfortunately, as yet unavailable. One possible approach to addressing therapeutically the problem of Gram-negative sepsis has been to target LPS itself by the use of an agent that would bind to, and sequester it. It has been shown by total synthesis¹⁸⁻²¹ that the toxicity of LPS resides in its structurally highly conserved glycolipid component called Lipid A.^{22;23} Lipid A is composed of a hydrophilic, bis-phosphorylated diglucosamine backbone, and a hydrophobic domain of 6 (E. coli) or 7 (Salmonella) acyl chains in amide and ester linkages²⁴⁻²⁶ (FIG. 1). The anionic and amphiphilic nature of lipid A (FIG. 1) enables it to bind to numerous substances that are positively charged and also possess amphipathic character. Over the past decade, there have been efforts involved in characterizing the interactions of lipid A with a number of classes of cationic amphipathic molecules including proteins,^{27;28} peptides,²⁹⁻³³ pharmaceutical compounds,^{34;35} and other synthetic polycationic amphiphiles.³⁶⁻³⁸ Importantly, from these and currently ongoing studies, it has been determined the pharmacophore necessary for optimal recognition and neutralization of lipid A³⁵ by small molecules requires two protonatable positive charges so disposed that the distance between them are equivalent to the distance between the two anionic phosphates on lipid A (˜14 Å), enabling ionic H-bonds between the phosphates on the lipid A backbone and the positive charges on the compound. In addition, appropriately-positioned pendant hydrophobic functionalities are necessary to further enhance binding affinity and stabilize the resultant complexes via hydrophobic interactions with the polyacyl domain of lipid A (for a recent review, see Ref. 39). These structural requisites were first identified in certain members of a novel class of compounds, the lipopolyamines, which were originally developed, and are currently being used as DNA transfection (lipofection) reagents.⁴⁰⁻⁴³ Compounds of the conjugated spermine class are of particular interest because they are active in vivo and afford protection in animal models of Gram-negative sepsis, are synthetically easily accessible, and, importantly, are nontoxic, on account of their degradation to physiological substituents (spermine and fatty acid).^{37;44}

### SUMMARY

Ongoing research by the present inventors seeks to systematically identify structural variations in the polyamine backbone that would impart additional, enthalpically-driven H-bond/van der Waals interactions. The polyamine amides such as lysine-spermine derivatives described herein exemplify a group of compounds that incorporate stereogenic H-bond donor/acceptor functionalities at one end of the polyamine scaffold. This confirms the obligatory requirement of a terminally-placed long-chain hydrophobic group for optimal endotoxin sequestration. The present inventors have also found significant differences in both the binding affinity and neutralization potency of L- and D-lysine conjugates. This suggests that an iterative substitution of the polyamine backbone with H-bond donor/acceptor functionalites with appropriate stereochemistry leads to yield highly potent, yet nontoxic endotoxin neutralizers. Examples of compounds contemplated as potent, yet nontoxic endotoxin neutralizers according to this disclosure are disclosed in US patent publication 20030187276 A1(U.S. Ser. No. 10/296,259) and PCT publication WO02/053519 A2, disclosures of which are incorporated herein by reference.

The present disclosure relates to a method for treating endotoxic shock condition or for inhibiting at least one of NO activity, TNF-α production, IL-6 production and cytokine activity by administering to a host in need thereof an effective amount of at least one compound represented by the formula: 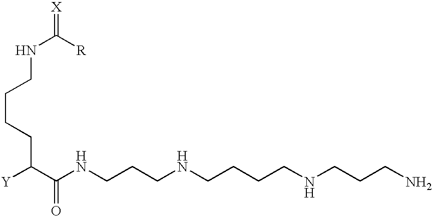
wherein X is O or H, H; R is a hydrophobic C₁₂-C₂₀ chain and Y is —NH₂ or —H, and pharmaceutically acceptable salts thereof and prodrugs thereof.

The present disclosure also relates to novel compounds of the above formula wherein Y is —H, pharmaceutically acceptable salts thereof and prodrugs thereof.

Other objects, features, and advantages of the present disclosure will become apparent from the following detailed description. It should be understood; however, that the detailed description and specific examples, while indicating preferred embodiments of the present invention, are given by way of illustration only and various modifications may naturally be performed without deviating from the present disclosure.

### BRIEF DESCRIPTION OF DRAWINGS

FIG. 1 illustrates the Structure of Lipid A, the toxic moiety of bacterial lipopolysaccharide.

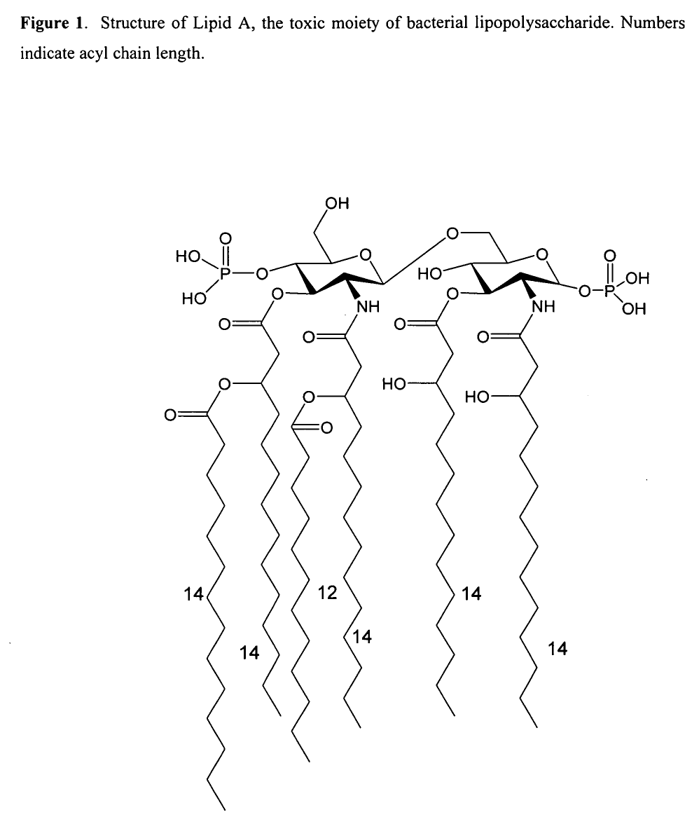

FIG. 2 is a graph illustrating the Binding affinity of compounds to LPS determined by the BODIPY-Cadaverine displacement method.

FIG. 3 is a graph illustrating the Nitric oxide (NO) inhibition in murine J774.A1 cells.

FIG. 4 is a graph illustrating the Correlation of NO inhibitory potency with carbon-length of straight-chain acyl/alkyl analogs.

FIG. 5 is a graph illustrating the Correlation of binding affinity of the Lys-spermine analogs (ED₅₀) determined by BC fluorescent probe displacement, with NO inhibition (IC₅₀) in murine J774 cells.

FIG. 6 is a chart illustrating lysine-spermine compounds binding to LPS isolated from diverse Gram-negative bacteria.

FIG. 7 are graphs illustrating the Inhibition by select Lys-spermine compounds of proinflammatory cytokines TNF-α and IL-6 in human blood stimulated with 10 ng/ml E. coli 0111:B4 LPS.

FIG. 8 illustrates a scheme for the synthesis of compounds employed pursuant to this disclosure.

### BEST AND VARIOUS MODES

The present disclosure relates to a method for treating endotoxic shock condition or for inhibiting at least one of NO activity, TNF-α production, IL-6 production and cytokine activity by administering to a host in need thereof an effective amount of at least one compound represented by the formula: 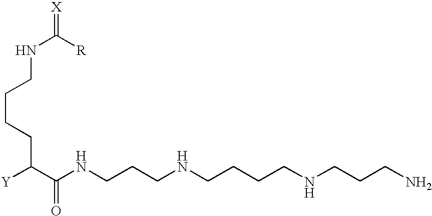
wherein X is O or H, H; R a hydrophobic C₁₂-C₂₀ chain and Y is —NH₂ or —H, and pharmaceutically acceptable salts thereof and prodrugs thereof.

The present disclosure also relates to novel compounds of the above formula wherein Y is —H, pharmaceutically acceptable salts thereof and prodrugs thereof.

Examples of hydrophobic C₁₂-C₂₀ chains are aliphatic groups, acyl groups, phenybenzyl, and groups with a OSO group in the a position. The aliphatic group can be saturated or ethylenically unsaturated, straight, cyclic or branched chain. The method of the present disclosure can be used in treating sepsis, inflammation and infections.

Prodrug forms of the compounds bearing various nitrogen functions (amino, hydroxyamino, hydrazino, guanidino, amidino, amide, etc.) may include the following types of derivatives where each R group individually may be hydrogen, substituted or unsubstituted alkyl, aryl, alkenyl, alkynyl, heterocycle, alkylaryl, aralkyl, aralkenyl, aralkynyl, cycloalkyl or cycloalkenyl groups as defined above.

Carboxamides, —NHC(O)R

Carbamates, —NHC(O)OR

(Acyloxy)alkyl Carbamates, NHC(O)OROC(O)R

Enamines, —NHCR(═CHCRO₂R) or —NHCR(═CHCRONR₂)

Schiff Bases, —N═CR₂

Mannich Bases (from carboximide compounds), RCONHCH₂NR₂

Preparations of such prodrug derivatives are discussed in various literature sources (examples are: Alexander et al., J. Med. Chem. 1988, 31, 318; Aligas-Martin et al., PCT WO pp/41531, p.30). The nitrogen function converted in preparing these derivatives is one (or more) of the nitrogen atoms of a compound of the invention.

Prodrug forms of carboxyl-bearing compounds of the disclosure include esters (—CO₂R) where the R group corresponds to any alcohol whose release in the body through enzymatic or hydrolytic processes would be at pharmaceutically acceptable levels.

Another prodrug derived from a carboxylic acid form of the disclosure may be a quaternary salt type

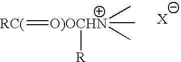

of structure described by Bodor et al., J. Med. Chem. 1980, 23, 469.

It is of course understood that the compounds of the present invention relate to all optical isomers and stereo-isomers at the various possible atoms of the molecule.

The compounds of this disclosure form acid and base addition salts with a wide variety of organic and inorganic acids and bases and includes the physiologically acceptable salts which are often used in pharmaceutical chemistry. Such salts are also part of this invention. Typical inorganic acids used to form such salts include hydrochloric, hydrobromic, hydroiodic, nitric, sulfuric, phosphoric, hypophosphoric and the like. Salts derived from organic acids, such as aliphatic mono and dicarboxylic acids, phenyl substituted alkonic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, β-hydroxybutyrate, butyne-1,4-dioate, hexyne-1,4-dioate, cabrate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toleunesulfonate, xylenesulfonate, tartarate, and the like.

Bases commonly used for formation of salts include ammonium hydroxide and alkali and alkaline earth metal hydroxides, carbonates, as well as aliphatic and primary, secondary and tertiary amines, aliphatic diamines. Bases especially useful in the preparation of addition salts include sodium hydroxide, potassium hydroxide, ammonium hydroxide, potassium carbonate, methylamine, diethylaamine, and ethylene diamine.

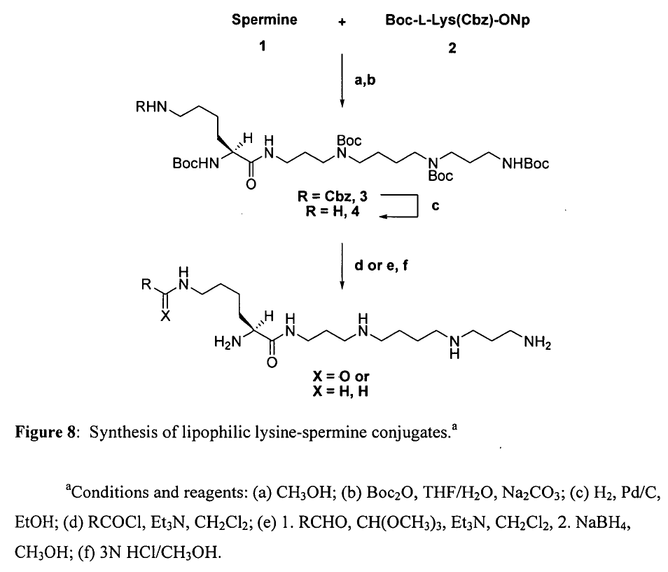

The method to be used for the synthesis of lysine-spermine conjugates enabled selective functionalization of the E-nitrogen atom of lysine and chromatographic purification prior to exposure of the extremely polar amine groups. Specifically, blockage of the polar amino groups on the polyamine conjugates uses Boc-carbamates, allowing normal-phase SiO₂ chromatography instead of the more time-consuming ion-exchange method previously reported.⁴⁵ Synthesis of these analogs, shown in FIG. 8, begins by coupling the free base of spermine 1 with either the L- or D-stereoisomer of the orthogonally-protected, active ester Boc-Lys(Cbz)-ONp 2. Dropwise addition of the active ester to a solution of spermine gives the statistical distribution of mono-, di- and un-substituted products. Reaction of the remaining unsubstituted amino groups of spermine with an excess of Boc₂O produced the per-Boc mixture. The resulting mixture can now be separated by standard silica gel chromatography. The purified mono-acylated derivative 3 is then subjected to catalytic hydrogenation in order to remove the Cbz-protecting group and gain the free amino intermediate 4. Use of ketone-free ethanol is desirable during this hydrogenation in order to prevent formation of a higher R_{f}, alkylated side-product. The amine 4 is then functionalized by standard acylation or reductive alkylation conditions to produce the protected forms of the Lys-spermine analogs. For the mono-alkyl derivatives, the imines are pre-formed then reduced using NaBH₄. In the case of dialkylated analogs 30 and 38, excess aldehyde is used in a reductive amination reaction with NaBH₃CN. In several cases unique functional groups are synthesized using common reaction conditions or are from commercial sources. The derivatized intermediates are purified using SiO₂ chromatography, and the Boc-groups removed using 3N HCl in MeOH to afford the Lys-spermine analogs in their HCl salt forms. Compounds are characterized by TLC, ¹H and ¹³C NMR, elemental analysis and LC/MS and all spectra are consistent with structures assigned.

Structure-activity Relationships: Binding Affinity and in vitro Neutralization Potency.

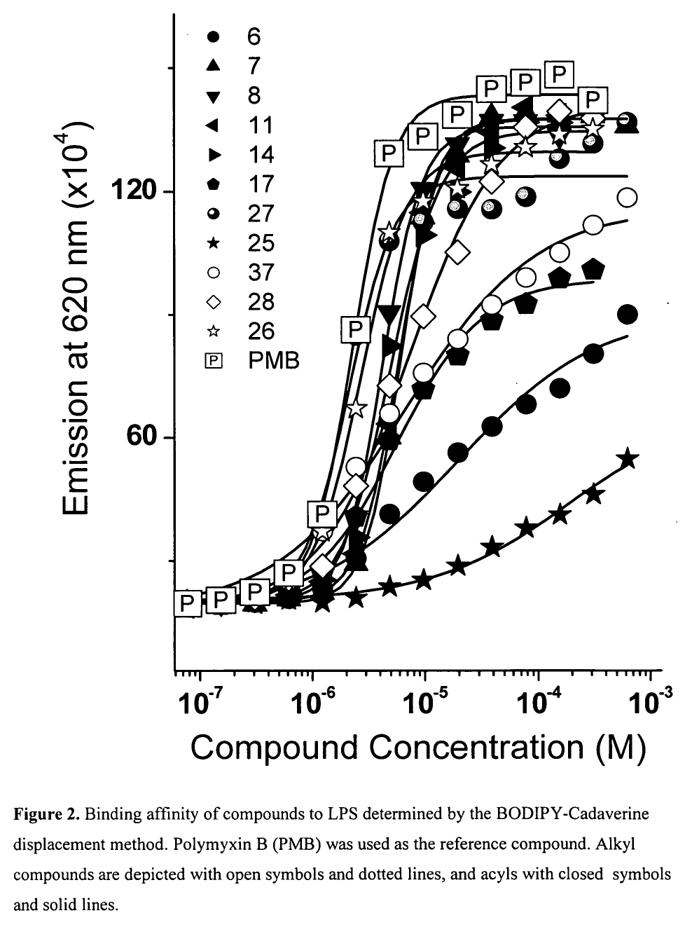

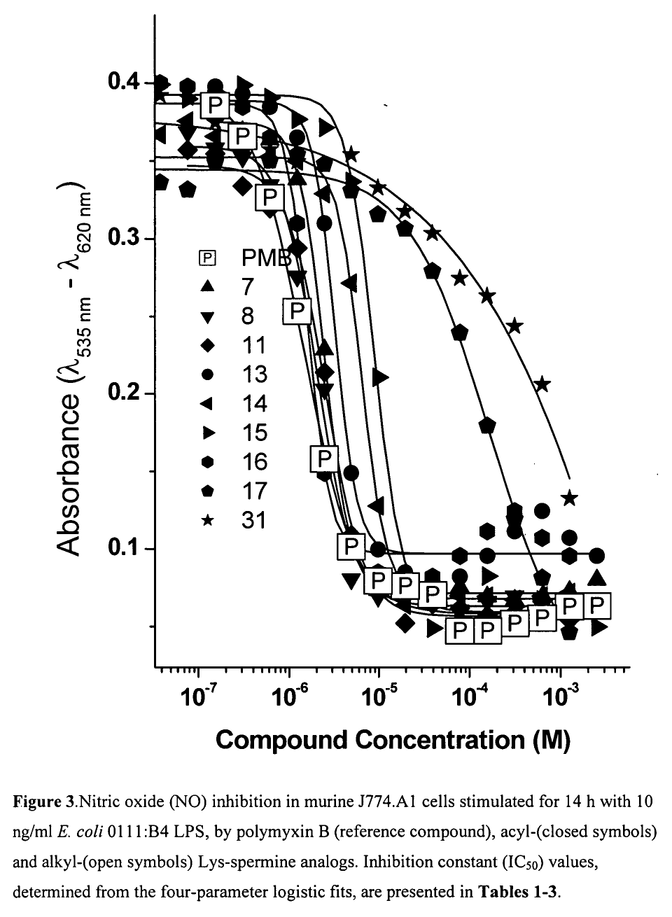

The relative binding affinities of the Lys-spermine analogs are examined with a recently-described⁴⁶ high-throughput fluorescence based displacement assay, using BODIPY-TR cadaverine (BC), and are reported as half-maximal effective displacement of probe (ED₅₀) in FIG. 2, and Tables 1-3. Murine monocytes (J774.A1 cells) produce measurable quantities of NO on exposure to LPS and provide a model for the rapid assessment of compounds to neutralize LPS activity. Compounds that neutralize LPS inhibit NO production in a dose-dependant manner from which 50% inhibitory concentrations (IC₅₀) can be determined, as shown in FIG. 3, and Tables 1-3. In all experiments, Polymyxin B (PMB), a decapeptide antibiotic, known to bind and neutralize LPS,^{29;47;48} is used as a reference compound.

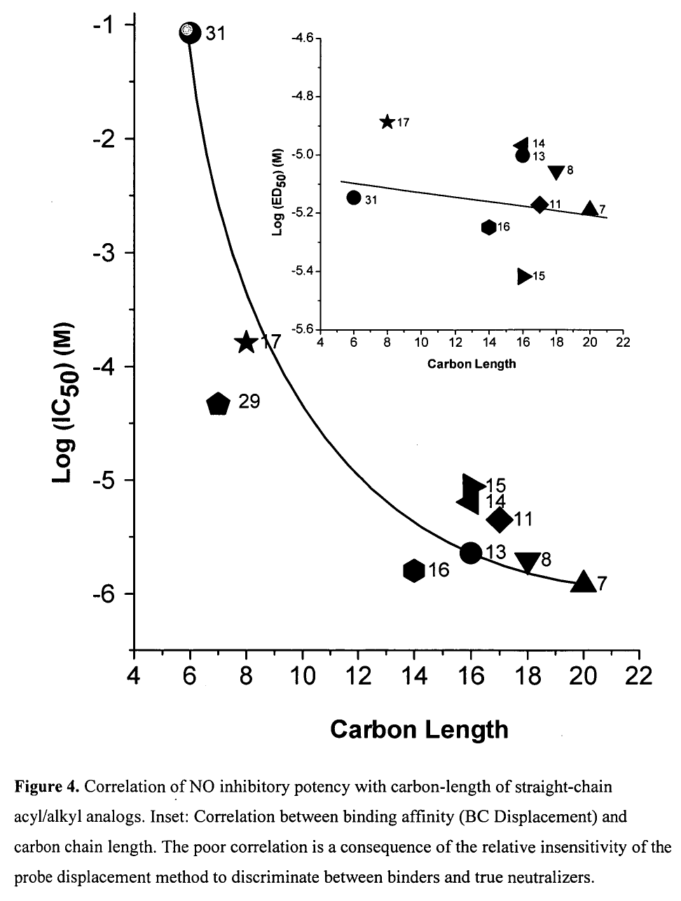

Hydrocarbon chain length. Lysine-spermine analogs with an unsubstituted ε-amino lysine, 5 (L-Lys, ED₅₀:40 μM), and 6 (D-Lys, ED₅₀: 58 μM) show poor binding in the displacement assays, and negligible inhibition of LPS-induced NO production. Substitution of the ε-amino group of lysine manifests in an increase in affinity (Table 1), but no striking correlation between hydrocarbon chain-length and affinity is evident (FIG. 4, inset). In contrast, increasing carbon chain length is clearly correlated to the potency of inhibition of LPS activity (FIG. 4). This apparent discordance is attributable to the displacement of LPS-bound BC being relatively insensitive to hydrophobic substituents, and dominated by electrostatic interactions.⁴⁶ The consequence of this limitation is the lack of discrimination between ligands that merely bind LPS, and those that truly neutralize LPS activity. All promising leads are also screened in NO inhibition assays. Chain lengths were critical determinants for NO-inhibiting activity as shown by the alkyl homologs C₆ 31 (>1000 μM), C₇ 29 (46 μM), Δ11-C₁₆ 27 (0.66 μM), as well as the acyl series from C₈ 17 (160 μM) and to C₂₀ 7 (1.2 μM).

Chain unsaturation. Trans unsaturation of the acyl chain is found to increase binding as shown by comparing Δ9-L-Lys-C₁₆ 15 (3.8 μM) with L-Lys-C₁₆ 14 (11 μM), and Δ11-L-Lys-C₁₈ 10 (4.2 μM) with L-Lys-C₁₆ 9 (16 μM). Similarly for the alkyls, the cis-unsaturated L-Lys-Δ11-C₁₆ 27 displays a higher IC₅₀ of 2.6 μM compared to its saturated counterpart, C₁₆ 26 (5.6 μM). However, this is not paralleled by an improvement in LPS-neutralizing activity; for instance, L-Lys-C₁₆ 14 (IC₅₀: 6.4 μM), and the fully saturated 15 (IC₅₀: 8.8 μM) are equipotent. The present inventors surmise, but are not bound thereby, that the observed enhanced affinity with the unsaturated analogues may also be an artifactual consequence of the probe displacement method. The unsaturated compounds are, in general marginally more water soluble than their saturated homologs, and thus may exhibit a higher effective local concentration at the LPS-bulk solvent interface.³⁴ It is to be noted that in vitro bioassays, as well as in animal models, the problem of differential solubility is mitigated by the presence of physiological concentrations of albumin which serve to solubilize both LPS and ligand.²⁸ Unsaturation of the hydrophobic substituent, therefore, while not expected to result in higher potency compounds, is a potentially useful strategy that might be of interest for evaluating the enhancing of the solubility of other, less-soluble analogues.

Steric interactions. Although analogues with short bulky substituents show increased binding with increasing chain carbon number, for instance, isobutyl 35 (101 μM) with 32 (9.6 μM) derived from (S)-(−)-citronellal, and bis-alkylated methylcyclohexyl 38 (4.0 μM) with the mono-alkylated methylcyclohexyl 37 (9.8 μM) (Table 2), none of these compounds are potent LPS neutralizers, as are the di- and tri-ether homologs 24 and 25 (IC₅₀: >1000 mM) and the polyethylene glycol polymer 23 (320 μM). The biphenyls 39 and 21 and anthracene 22 all yield reasonably high LPS affinities (ED₅₀: 3.7 μM, 7.9 μM, and 7.1 μM, respectively) but are poor inhibitors of LPS bioactivity (IC₅₀: >100 μM). These results emphasize the obligatory requirement for long-chain aliphatic hydrocarbon substituents for optimal biological potency.

Stereochemistry of Lys residue. Inverting the stereochemistry of the α-carbon of lysine do not cause any appreciable effect on binding affinity for the stereoisomeric pair D-Lys-C₁₆ 13 (10 μM) and L-Lys-C₁₆ 14 (11 μM), but a distinct enhancement for longer chain D-Lys-C₁₈ 8 (8.8 μM), as compared to L-Lys-C₁₈ 9 (16 μM). This is consistent with the higher potency for the D-analogues in inhibition NO production. The lipid A moiety is a chiral, and the mode of binding may be effected by the configuration of asymmetric centers.

Alkylation versus Acylation. Alkyl compounds bind more strongly than their acyl equivalents; compare, for example: alkyl C₁₆ 26 (5.6 μM) and acyl C₁₆ 14 (11 μM). This may be attributable to the loss of a protonatable positive charge on acylating the i-amino group, leading to poorer solubility, as mentioned earlier.

Comparison of IC₅₀ and ED₅₀:

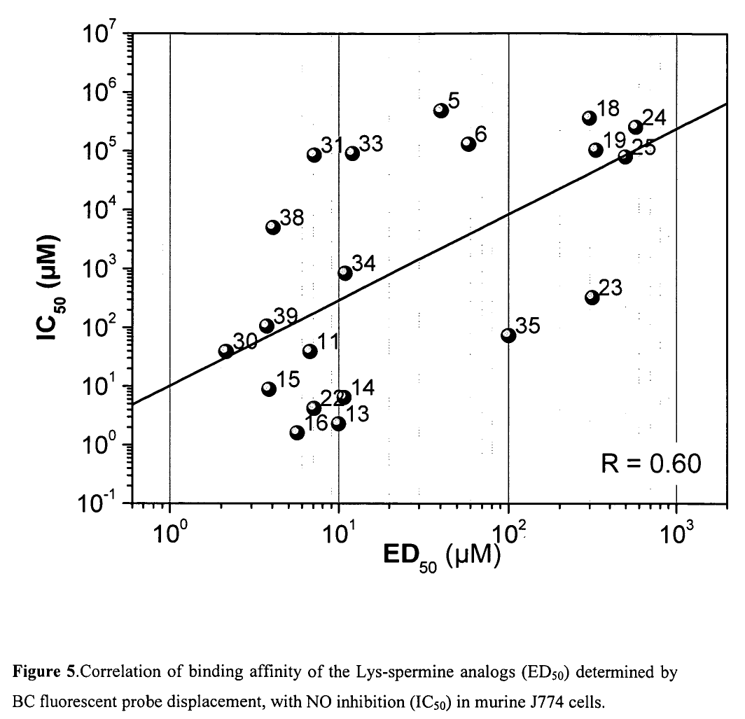

The graph of IC₅₀ vs. ED₅₀ values display a linear trend with a correlation coefficient of R=0.64 (FIG. 5). LPS binders with strong hydrophobic interactions strayed from linearity due to the BC-LPS displacement assay not accurately predicting hydrophobic interactions which have been shown to be crucial for LPS neutralization. This is seen also for the aromatic and bulky substituents which were relatively bereft of biological activity in contrast to their high binding affinities and so appeared as a cluster in the upper left hand side of the IC₅₀ vs. ED₅₀ graph (FIG. 5).

Comparison of LPS From Different Gram Negative Bacteria:

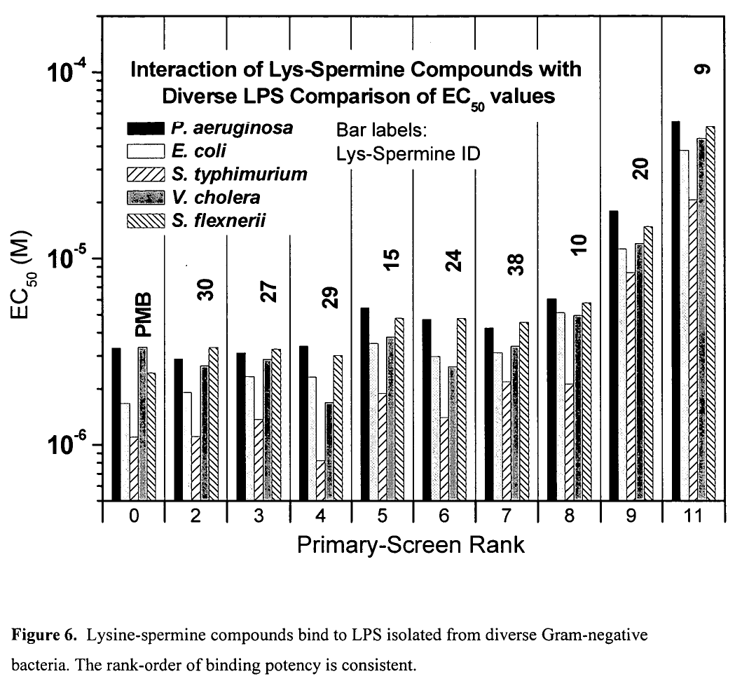

Although the structure of lipid A is highly conserved among Gram-negative bacteria, the polysaccharide domain is highly diverse among Gram-negative bacteria.^{49;50} Since the Lys-spermine library was designed to bind to the conserved lipid A portion, we expected that there would be little variation in binding to a diverse range of LPS from different bacteria. As shown in FIG. 6, the highest affinity Lys-spermine analogs were shown to consistently bind to LPS from different bacteria in the 1-10 μM region and the relatively poor binders bound to all the LPS in the 10-100 μM range. This clearly shows that the Lys-spermine compounds bind to a variety of LPS structures, and thus may be clinically useful.

Dose-dependent Inhibition of Proinflammatory Cytokines in Human Whole Blood, Determined by Multiplexed Cytometric Bead Assay:

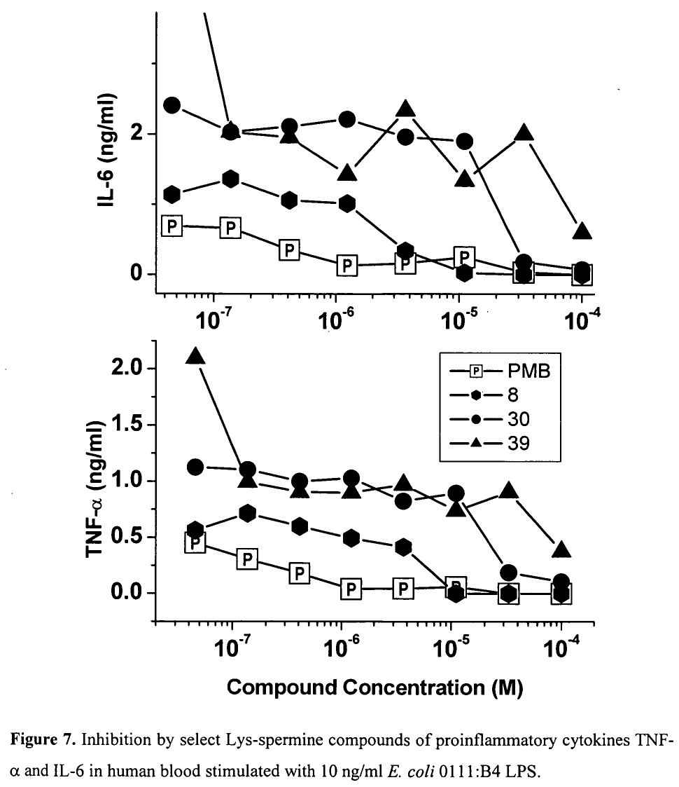

Having verified that the Lys-spermine compounds are active in inhibiting NO production in murine macrophages, independent confirmation that they would also inhibit LPS-induced inflammatory responses in human cells is carried out. The activity of a subset of active Lys-spermine compounds is examined for their ability to inhibit TNF-α and IL-6 production in whole human blood, stimulated ex vivo with LPS. As shown in FIG. 7, the rank-order of the inhibitory potencies in this assay generally parallels NO inhibition activity, 8 being almost as potent as polymyxin B, the reference compound.

Protective Effects in a Mouse Model of Endotoxic Shock:

Based on the results of the displacement assays, NO and cytokine inhibition data, 8 is elected for detailed evaluation in animal experiments. The LD₁₀₀ (lethal dose—100%) dose is determined to be—100 ng per mouse (female, outbred, CF-1 mice, sensitized with 800 mg/kg D-galactosamine). In all experiments reported herein, a supralethal dose of 200 ng per mouse, in a final volume of 0.2 ml saline is used. The dose-response of protection afforded by 8 is depicted in Table 4. Previous studies with labile spermine conjugates such as DOSPER³⁷ had shown the window of protection to be very short, a 15 minute window of protection. Compound 8, with its greater anticipated hydrolytic stability, is examined to see if it affords a more extended time-window of protection. 200 μg of 8 in a final volume of 0.2 ml injections are administered intraperitoneally at times of −6, −4, −2, 0, +1, and +2 relative to time-zero, the time at which all mice are challenged with 200 ng/mouse LPS injections. Compound 8 provides significant protection up to 6 h prior to LPS challenge (Table 5). Based on these results, another time-course experiment with subcutaneous, rather than i.p. injections is undertaken with a much longer time window (−24, −16, −12, −8, −4, 0, and +2 hours relative to the time of LPS administration). Testing to see if in this treatment regime, which is characterized by a slow, gradual systemic absorption from the site of injection, a longer duration of protection would be observed is carried out. Lethality is once again assessed 24 hours following the final injection. Two of the 5 mice in the −24 cohort survive, as do 3 of the 5 in the −16, −12, and −8 cohorts (Table 6), indicating significant protection even when the compound is administered 16 h ahead of LPS challenge. These results indicate a significantly prolonged temporal window of protection compared to DOSPER.³⁷

A focused library of alkyl or acyl c-substituted lysine-spermine conjugates is synthesized with even carbon-numbered chains of C₁₄ to C₂₀ lengths. These analogs and their associated LPS-binding, NO inhibition and NFκB inhibition activities are shown in Table 7. These data clearly show high potency compounds are those that have chain lengths about C₁₈. Furthermore, the data showhigh activity compounds are those with chain lengths between C₁₆ and C₂₀. The data show that high activity compounds could be acyl (X═O) substituted. The data show that high activity compounds could be alkyl (X═H, H) substituted. The exemplary compounds L-Lys-ε-(stearoyl)-N¹-spermine, D-Lys-ε-(stearoyl)-N¹-spermine, L-Lys-ε-(octadecanyl)-N¹-spermine and D-Lys-ε-(octadecanyl)—N1-spermine all show high activity for the prevention of LPS-induced NFκβ cytokine release from stimulated lymphocytes. Furthermore, the exemplary compounds L-Lys-ε-(stearoyl)-N¹-spermine, D-Lys-ε-(stearoyl)-N¹-spermine, L-Lys-ε-(octadecanyl)-N¹-spermine and D-Lys-ε-(octadecanyl)-N¹-spermine all show high activity for the prevention of LPS-induced NO release from stimulated lymphocytes.

In conclusion, the interactions of a focused library of lysine-spermine conjugates with Gram-negative bacterial lipopolysaccharides have been characterized. Lysine-spermine conjugates with the ε-amino terminus of the lysinyl moiety derivatized with long-chain aliphatic hydrophobic substituents(e.g. C₁₂-C₂₀) in acyl or alkyl linkage bind to the lipid A moiety of LPS, and neutralize their toxicity. The presence of long-chain aliphatic hydrophobic functionalities seems important for biological activity. The utilization of nontoxic and ubiquitous building blocks (spermine, lysine, and long-chain fatty acid) in the synthesis of these compounds would predict low systemic toxicity, and are therefore desirable for providing novel therapeutic agents aimed at the prevention or treatment of endotoxic shock states.

The following non-limiting examples are presented to further illustrate the present disclosure:

### EXAMPLE 1

### General Synthetic Methods

The sources of all chemical reagents and starting materials are of the highest grade available and are used without further purification. Thin-layer chromatography analysis and column chromatography is performed using Merck F₂₅₄ silica gel plates and Baker 40 μm flash chromatography packing, respectively. TLC analysis uses the following solvent systems with detection by ninhydrin staining: a) hexane/ethyl acetate/methanol 48:48:4; b) 2-propanol/pyridine/glacial acetic acid/H₂O, 4:1:1:2; c) CHCl₃/MeOH/NH₄OH 85:15:1. LC/MS analyzes are performed using a Gilson 322 HPLC system coupled to a 215 liquid handler. Retention of these polar molecules on C-18 reverse-phase HPLC media is facilitated by the use of 0.05% heptafluorobutyric acid as an ion-pairing reagent in the mobile phase. This allows analysis of the compounds in their underivatived forms.

Detection is by a Finnigan AQA operating in ESI⁺ mode (m/z range 140 to 1600 amu) together with an Agilent 1100 series DAD detector (UV range 220 to 320 nm). Gradient elution from 2 to 7 min. is performed using 2% to 100% CH₃CN in H₂O (both with 0.05% heptafluorobutyric acid added as the volatile ion-pairing reagent). ¹H and ¹³C NMR spectra are recorded at 500 MHz and 125.8 MHz, respectively on a Brucker WM500 spectrometer at the University of Washington, Seattle. ¹H NMR signals are generally multiples unless otherwise noted as s=singlet, d=doublet or t=triplet. Chemical shifts are relative to external 3-(trimethylsilyl)-1-propanesulfonic acid, sodium salt.

The method for the synthesis of lysine-spermine conjugates enables selective functionalization of the i-nitrogen atom of lysine and chromatographic purification prior to exposure of the extremely polar amine groups. Specifically, blockage of the polar amino groups on the polyamine conjugates uses Boc-carbamates, allowing normal-phase SiO₂ chromatography instead of the more time-consuming ion-exchange method previously reported.⁵¹ Synthesis of these analogs begins by coupling of the free base of spermine 1 with the orthogonally-protected L- or D-stereoisomeric forms of Boc-Lys(Cbz)-ONp active ester 2. Dropwise addition of the active ester to a solution of spermine gives the statistical distribution of mono-, di- and un-substituted products. Reaction of the remaining unsubstituted amino groups of spermine with an excess of Boc₂O produced the per-Boc mixture. The resulting mixture can now be separated by standard silica gel chromatography.

The purified mono-acylated derivative 3 is then subjected to catalytic hydrogenation in order to remove the Cbz-protecting group and gain the free amino intermediate 4. Use of the ketone-free ethanol during this hydrogenation is advantageous in order to prevent formation of a higher R_{f}, alkylated side-product. The amine 4 is then functionalized by standard acylation or reductive alkylation conditions to produce the protected forms of the analogs 5. For the mono-alkyl derivatives, the imines are pre-formed then are reduced using NaBH₄. In the case of dialkylated analogs 30 and 38, excess aldehyde is used in a reductive amination reaction with NaBH₃CN. In several cases unique functional groups are synthesized using common reaction conditions or are from commercial sources. The derivatized intermediates 5 are purified using SiO₂ chromatography. Removal of the Boc-groups using 3N HCl in MeOH gives the desired materials in their HCl salt forms. Compounds are characterized by TLC, ¹H and ¹³C NMR, elemental analysis and LC/MS and all spectra are consistent with structures assigned.

### EXAMPLE 2

### Synthetic Methods for Precursor Compounds

Boc-L-Lys(Cbz)-N¹-spermine-Boc₃, (3)—To a stirred solution of spermine 1 (11.30 g, 1.4 eq, free base form) in MeOH (200 mL) is added dropwise over 1.5 h the active ester 2 (20.0 g, 40 mmole) in MeOH (200 mL) at room temp. After this dropwise addition, TLC analysis (b) shows that the expected mixture of products is formed (di-substituted side-product R_{f}=0.76; mono-substituted desired product R_{f}=0.50 and un-substituted spermine R_{f}=0.08). If the optimal ratio is not produced additional active ester in MeOH is added dropwise. After stirring for 2 h, the solvent is evaporated to give a yellow solid that is suspended in THF (300 mL) and H₂O (100 mL). A solution of di-tert-butyl carbonate (43.5 g, 5.0 eq) in tetrahydrofuran (50 mL) is added at room temperature. The pH is adjusted periodically to ˜10 with a 10% Na₂CO₃ solution. A precipitate is noted after 10 minutes. After stirring for 18 h, TLC analysis (a) shows that the expected products are formed (elution order had inverted from that given above). Most of the THF is evaporated in vacuo. The resulting mixture is dissolved in EtOAc (400 mL) and H₂O (400 mL). The organic layer is removed and the aqueous layer is re-extracted with EtOAc (3×400 mL). The combined organic layers are washed with ice-cold 0.1 N HCl (2×250 mL) followed by brine. The organic layer is dried over MgSO₄, filtered and concentrated to give a crude oil which is purified via silica gel chromatography (column dimensions 8×17 cm) using stepwise elution with 1:1 hexanes/EtOAc containing 0%, 2%, 3%, 4% and 5% MeOH (1L each). The order of elution is Boc₄-spermine (25% yield (spermine can be recovered after acid deprotection and conversion to the free base)), the desired mono-substituted Boc-Lys(Cbz)-spermine-Boc₃ 3 (19.4 g, 56% yield) and finally eluting last is the di-substituted side-product. ¹H NMR of the desired product shows this to be a mixture of cis- and trans-carbamate rotomers. It is used in the next reactions without further characterization.

### EXAMPLE 3

### Boc-L-Lys-N¹-spermine-Boc₃

(4)—To a stirred solution of the orthogonally protected lysine-spermine conjugate 3 (19.4 g, 22.5 mmole) in EtOH (200 mL, ketone and aldehyde free EtOH) is added palladium 10 wt. % on activated carbon (10.0 g) in a round-bottom flask. The reaction flask is purged 3× with H₂ is then placed under 5 psi H₂ pressure. After stirring for 4.0 h at room temperature, TLC analysis (c) shows the reaction is complete. An extra amount of activated charcoal is added to the mixture and the catalyst is removed by filtering over a pad of Celite. The pad is washed with EtOH (2×50 mL) and the combined filtrates are evaporated to give 4 as a white foam in quantitative yield. Following evaluation by the above TLC system this product are used directly in the next examples.

### EXAMPLE 4

### Representative Acylation Reaction

L-Lys(palmitoyl)-N¹-spermine (14)—To the amine precursor 4 (9.66 g, 13.22 mmol) is added Et₃N (5.5 mL, 3.0 equiv) and dry CH₂Cl₂ (100 mL) via a syringe under an atmosphere of argon. The resulting solution is chilled to 0° C. in an ice bath and palmitoyl chloride (6.0 mL, 1.5 equiv) is added via a syringe. After stirring under an argon atmosphere overnight TLC analysis (c) shows that the expected product is formed. The solution is diluted in CH₂Cl₂ (100 mL) and H₂O (100 mL). The organic layer is removed and the aqueous layer is extracted twice more with CH₂Cl₂ (2×100 mL). The combined organic layer is extracted with ice cold 0.1N HCl (100 mL) then brine and dried over MgSO₄, filtered and concentrated to give the crude oil. This is purified via silica gel chromatography (column dimensions 8×17 cm) using stepwise elution with hexanes/EtOAc 1:1 containing 0%, 2%, 3%, 4%, 5% and 6% MeOH (500 mL each) to give the Boc-protected product 13 as a clear oil (6.48 g, 51%). Removal of the protecting groups is accomplished by treating a stirred solution of the above product (6.48 g, 6.68 mmol) in MeOH (50 mL) with 6N HCl (50 mL) at room temperature. After 3 h TLC analysis (b) shows that the reaction is complete. The solvents are evaporated to give the desired product 14 in its 4HCl salt form as a white solid (4.78 g, 100%). TLC analysis (b); R_{f}=0.19. ¹H NMR (D₂O, δ): 3.93 (1H), 3.45 (1H), 3.03 (13H), 2.12 (2H), 2.02 (2H), 1.85 (4H), 1.75 (s, 4H), 1.43 (4H), 1.32 (2H), 1.11 (24H), 0.72 (t, 3H). ¹³C NMR (D₂O, ppm): 175.7, 169.8, 53.4, 46.7 (m), 45.2, 44.6, 38.8, 36.5, 36.1, 31.9, 30.4, 30.0 (m), 29.9 (m), 29.6 (m), 29.4, 28.2, 25.7, 25.5, 23.6, 22.8, 22.7, 22.3, 21.7, 13.8. LC/MS (ret time, 7.2 min), calcd for C₃₂H₆₈N₆O₂ m/z 568, obsd 569 (MH⁺). Anal. (C₃₂H₇₂Cl₄N₆O₂) C, H, N.

### EXAMPLE 5

### Representative Mono-alkylation Reaction

L-Lys(3,3-dimethyl-1-butane)-N¹-spermine (18)—To 0.58 g (0.82 mmol) of amine 4 in 5 mL of dry CH₂Cl₂ under argon is added 0.27 mL (3 eq) of trimethylorthoformate, 0.17 mL of Et₃N (1.5 eq) and 0.31 mL (3 eq) of 3,3-dimethylbutyraldehyde. The resulting solution is stirred at r.t. for 2 h when the solvents are evaporated. The oily residue is dissolved in 5 mL of CH₃OH and 70 mg (2 eq) of NaBH₄ is added. After 2 h the solvent is evaporated and the residue is partitioned between 0.01 N HCl and CH₂Cl₂ (50 mL each). The aqueous part is washed with an additional portion of CH₂Cl₂ and the combined organic layers are washed with brine, dried with MgSO₄ and evaporated to give 0.64 g crude oil. Column chromatography using CHCl₃/MeOH/concd NH₄OH 96:4:0.2 gives 0.31 g (64% yield) pure protected product. This is dissolved in 3 mL of CH₃OH and treated with 3 mL of 6N HCl at r.t. for 3 h. Evaporation gives 0.24 g (96% yield) of 18 as a white solid. TLC analysis (b); R_{f}=0.21. ¹H NMR (D₂O, δ): 3.95 (1H), 22 3.31 (2H), 3.05 (14H), 2.04 (2H), 1.88 (4H), 1.71 (6H), 1.53 (2H), 1.41 (2H), 0.88 (9H). LC/MS (ret time, 6.1 min), calcd for C₂₂H₅₀N₆O m/z 414, obsd 415 (MH⁺). Anal. (C₂₂H₅₅Cl₅N₆O 0.5H₂O) C, H, N.

### EXAMPLE 6

### Representative di-alkylation Reaction

L-Lys-ε-(bis-(n-heptyl))-N¹-spermine (30)—A solution containing 0.22 g (0.30 mmol) of amine 4, 0.42 mL (3 mmol, 10 eq) of n-heptanal and 0.19 g (3 mmol, 10 eq) of NaBH₃CN in 10 mL of CH₃OH is treated with glacial HOAc (5 drops). The pH is measured to be 4 by paper. Following overnight stirring the solvent is evaporated and the residue is partitioned between 1N NaOH and CH₂Cl₂ (50 mL each). An additional CH₂Cl₂ wash of the aqueous layer is performed and the combined organic layers are washed with brine, dried over MgSO₄ and evaporated to give 0.33 g crude product. Column chromatography using CHCl₃/MeOH/concd NH₄OH (96:4:0.2) to give 0.20 g (71% yield) pure protected product. This is dissolved in 1 mL of CH₃OH and treated with 1 mL of 6N HCl at r.t. for 3 h. Evaporation gives 0.11 g (73% yield) of 30 as a white solid. TLC analysis (b), R_{f}=0.21. ¹H NMR (D₂O, δ): 3.93 (t, 1H), 3.28 (2H), 3.04 (16H), 2.02 (2H), 1.85 (4H), 1.71 (s, 4H), 1.62 (6H), 1.40 (4H), 1.25 (14H), 0.81 (t, 6H). ¹³C NMR (D₂O, ppm): 175.7, 169.8, 53.4, 46.7 (m), 45.2, 44.6, 38.8, 36.5, 36.1, 31.9, 30.4, 30.0 (m), 29.9 (m), 29.6 (m), 29.4, 28.2, 25.7, 25.5, 23.6, 22.8, 22.7, 22.3, 21.7, 13.8. LC/MS (ret time, 7.3 min), calcd for C₃₂H₆₈N₆O₂ m/z 568, obsd 569 (MH⁺).

### EXAMPLE 7

### Representative Individual Analogs

L-Lys-N¹-spermine (5)¹—TLC analysis (b); R_{f}=0.04. LC/MS (ret time, 5.5 min), calcd for C₁₆H₃₈N₆O m/z 330, obsd 331 (MH⁺).

D-Lys-N¹-spermine (6)—Synthesis of analog 6 uses Boc-D-Lys(Boc)-ONp in place of the orthogonally protected lysine derivative that is used for the synthesis of 14. Coupling with spermine followed by protection of the remaining amino groups as their Boc-carbamates gives the protected intermediate following purification by column chromatography. Deprotection using 6N HCl in CH₃OH gives the desired product 6. TLC analysis (b); R_{f}=0.04. ¹H NMR (D₂O, δ): 3.92 (t, 1H), 3.29 (2H), 3.07 (10H), 2.93 (t, 2H), 2.04 (2H), 1.84 (4H), 1.72 (4H), 1.54 (2H), 1.34 (2H). ¹³C NMR (D₂O, ppm): 168.7, 52.2, 45.8 (m), 44.2, 43.6, 40.0, 37.8, 35.4 (m), 29.0, 25.4, 24.6, 22.6, 21.8 (m), 20.2. LC/MS (ret time, 5.5 min), calcd for C₁₆H₃₈N₆O m/z 330, obsd 331 (MH⁺). HRMS m/z calcd for C₁₆H₃₈N₆O (M+H) 331.3185, found 331.3173. L-Lys-ε-(eicosanoyl)-N¹-spermine (7)—TLC analysis (b); R_{f}=0.08. ¹H NMR (D₂O, δ): 3.94 (1H), 3.48 (1H), 3.06 (13H), 2.15 (2H), 2.06 (2H), 1.88 (4H), 1.75 (4H), 1.47 (4H), 1.36 (2H), 1.16 (32H), 0.77 (3H). ¹³C NMR (D₂O, ppm): 175.5, 169.8, 53.4, 46.9 (m), 45.6, 44.8, 38.8, 36.8 (m), 36.0, 31.9, 30.4, 29.7 (m), 29.5, 29.3, 28.5, 25.9, 25.7, 23.8, 23.2, 23.1, 22.8, 21.7, 13.8. LC/MS (ret time, 7.6 min), calcd for C₃₆H₇₆N₆O₂ m/z 625, obsd 626 (MH⁺).

D-Lys-ε-(stearoyl)-N¹-spermine (8)—TLC analysis (b); R_{f}=0.13. ¹H NMR (D₂O, δ): 3.94 (1H), 3.47 (1H), 3.06 (13H), 2.13 (2H), 2.04 (2H), 1.87 (4H), 1.75 (4H), 1.47 (4H), 1.36 (2H), 1.16 (28H), 0.79 (3H). ¹³C NMR (D₂O, ppm): 175.9, 170.1, 53.4, 47.1 (m), 45.5, 44.7, 39.0, 36.8 (m), 36.0, 31.9, 30.6, 29.8 (m), 29.6, 29.3, 28.4, 25.9, 25.7, 23.8, 23.2, 23.1, 22.8, 13.8. LC/MS (ret time, 7.4 min), calcd for C₃₂H₆₈N₆O₂ m/z 597, obsd 598 (MH⁺).

L-Lys-ε-(stearoyl)-N¹-spermine (9)—LC/MS (ret time, 7.4 min), calcd for C₃₄H₇₂N₆O₂ m/z 597, obsd 598 (MH⁺).

L-Lys-ε-(heptadecanoyl)-N¹-spermine (11)—TLC analysis (b); R_{f}=0.19. ¹H NMR (D₂O, δ): 3.96 (1H), 3.47 (1H), 3.08 (13H), 2.14 (2H), 2.04 (2H), 1.87 (4H), 1.78 (4H), 1.50 (4H), 1.36 (2H), 1.22 (26H), 0.78 (3H). ¹³C NMR (D₂O, ppm): 175.7, 169.9, 53.2, 47.2 (m), 45.4, 44.8, 39.0, 36.6 (m), 36.1, 31.9, 29.9 (m), 29.5, 29.3, 28.4, 25.8, 25.7, 23.8, 22.6, 22.5, 22.2, 22.0, 14.8. LC/MS (ret time, 7.2 min), calcd for C₃₃H₇₀N₆O₂ m/z 583, obsd 584 (MH⁺).

L-Lys-ε-(hexadecanesulfonamide)-N¹-spermine (12)—¹H NMR (D₂O, δ): 4.04 (1H), 3.53 (1H), 3.30 (1H), 3.22 (2H), 3.17 (14H), 2.18 (2H), 2.00 (4H), 1.82 (6H), 1.67 (2H), 1.52 (4H), 1.34 (22H), 0.95 (t, 3H). LC/MS (ret time, 7.3 min), calcd for C₃₂H₇₀N₆O₃S m/z 619, obsd 620 (MH⁺).

D-Lys-ε-(palmitoyl)-N¹-spermine (13)—TLC analysis (b); R_{f}=0.21. ¹H NMR (D₂O, δ): 3.94 (1H), 3.47 (1H), 3.06 (13H), 2.13 (2H), 2.04 (2H), 1.87 (4H), 1.75 (4H), 1.47 (4H), 1.36 (2H), 1.16 (24H), 0.78 (3H). ¹³C NMR (D₂O, ppm): 175.7, 169.8, 53.4, 47.2 (m), 45.6, 44.8, 39.0, 36.6 (m), 36.1, 31.9, 29.8 (m), 29.6, 29.3, 28.4, 25.9, 25.7, 23.8, 22.8, 23.1, 22.8, 22.1, 14.0. LC/MS (ret time, 7.2 min), calcd for C₃₂H₆₈N₆O₂ m/z 569, obsd 570 (MH⁺).

L-Lys-ε-(myristoyl)-N¹-spermine (16)—TLC analysis (b); R_{f}=0.22. ¹H NMR (D₂O, δ): 3.92 (1H), 3.27 (2H), 3.03 (14H), 2.12 (2H), 2.07 (4H), 1.83 (4H), 1.66 (6H), 1.48 (4H), 1.20 (20H), 0.78 (3H). LC/MS (ret time, 7.0 min), calcd for C₃₀H₆₄N₆O₂ m/z 541, obsd 542 (MH⁺).

L-Lys-ε-(octanoyl)-N¹-spermine (17)—TLC analysis (b); R_{f}=0.20. LC/MS (ret time, 5.7 min), calcd for C₂₁H₄₆N₆O₂ m/z 414, obsd 415 (MH⁺).

D-Lys-ε-(isopropoyl)-N¹-spermine (18)‘TLC analysis (b); R_{f}=0.24. ¹H NMR (D₂O, δ): 3.90 (1H), 3.28 (3H), 3.05 (13H), 2.40 (1H), 2.02 (2H), 1.82 (4H), 1.71 (s, 2H), 1.47 (2H), 1.28 (2H), 0.99 (6H). ¹³C NMR (D₂O, ppm): 180.8, 175.8, 53.2, 47.0 (m), 45.2, 44.6, 38.6, 36.4 (m), 35.1, 30.4, 28.1, 25.7, 23.8, 29.4, 22.8 (m), 21.6, 18.9. LC/MS (ret time, 5.5 min), calcd for C₂₀H₄₄N₆O₂ m/z 400, obsd 401 (MH⁺).

D-Lys-ε-(2-norbornaneacetoyl)-N¹-spermine (20)—TLC analysis (b); R_{f}=0.22. ¹H NMR (D₂O, δ): 3.88 (1H), 3.24 (2H), 3.05 (13H), 2.02 (4H), 1.80 (4H), 1.68 (4H), 1.33 (8H), 0.98 (5H). LC/MS (ret time, 6.0 min), calcd for C₂₅H₅₀N₆O₂ m/z 466, obsd 467 (MH⁺).

D-Lys-ε-(4-biphenycarboxamide)-N¹-spermine (21)—TLC analysis (b); R_{f}=0.13. ¹H NMR (D₂O, δ): 7.77 (6H), 7.43 (3H), 3.87 (1H), 3.48 (2H), 3.16 (2H), 2.95 (10H), 1.94 (2H), 1.83 (2H), 1.72 (2H), 1.62 (6H), 1.34 (2H). LC/MS (ret time, 6.3 min), calcd for C₂₉H₄₆N₆O₂ m/z 511, obsd 512 (MH⁺).

### EXAMPLE 7

L-Lys-ε-(4-(1-pyrene)-butanoyl)-N¹-spermine (22)—Synthesis of analog 22 is by acylation with 1-pyrenebutanoic acid succinimidyl ester from Molecular Probes, Eugene, Oreg. (cat # P-130). TLC analysis (b); R_{f}32 0.15. ¹H NMR (D₂O, δ): 7.34 (d, 1H), 7.22 (3H), 7.08 (2H), 6.98 (2H), 6.88 (d, 1H), 3.74 (t, 1H), 3.18 (1H), 3.01 (4H), 2.93 (2H), 2.86 (1H), 2.77 (1H), 2.72 (2H), 2.65 (2H), 2.56 (1H), 2.40 (2H), 1.97 (2H), 1.73 (2H), 1.68 (2H), 1.54 (6H), 1.40 (2H), 0.98 (4H). LC/MS (ret time, 6.6 min), calcd for C₃₆H₅₂N₆O₂ m/z 601, obsd 602 (MH⁺).

### EXAMPLE 8

L-Lys-ε-(methylpolyethyleneglycolpropionyl)-N¹-spermine (23)—The active ester to use to acylate the ε-nitrogen atom is mPEG-SPA (mw 2000) from Nektar Therapeutics (cat. No. 2M4MODO1). TLC analysis (b); R_{f}=0.24. ¹H NMR (D₂O, δ): 3.80 (1H), 3.50 (large OCH₂ envelope), 3.42 (6H), 2.92 (15H), 2.34 (1H), 1.96 (1H), 1.75 (4H), 1.62 (4H), 1.41 (1H), 1.23 (1H). LC/MS (ret time, 6.1 min), Obsd an envelope of m/z centered at 650.

L-Lys-ε-(2-[2-(2-methoxyethoxy)ethoxy]acetoyl)-N¹-spermine (24)—TLC analysis (b); R_{f}=0.11. ¹H NMR (D₂O, δ): 4.01 (3H), 3.91 (1H), 3.62 (6H), 3.31 (8H), 3.03 (12H), 2.06 (2H), 1.82 (6H), 1.53 (1H), 1.32 (1H). LC/MS (ret time, 5.4 min), calcd for C₂₃H₅₀N₆O₅ m/z 490, obsd 491 (MH⁺). L-Lys-ε-(2-(2-methoxyethoxy)acetoyl)-N¹-spermine (25)‘TLC analysis (b); R_{f}=0.09. ¹H NMR (D₂O, δ): 3.98 (3H), 3.92 (1H), 3.62 (6H), 3.31 (6H), 3.24 (6H), 3.03 (6H), 2.06 (2H), 1.87 (2H), 1.80 (4H), 1.53 (1H), 1.32 (1H). LC/MS (ret time, 5.7 min), calcd for C₂₁H₄₆N₆O₄ m/z 446, obsd 447 (MH⁺).

L-Lys-ε-(“hexadecyl)-N¹-spermine (26)—TLC analysis (b); R_{f}=0.11. ¹H NMR (D₂O, δ): 3.97 (1H), 3.48 (1H), 3.04 (15H), 2.04 (2H), 1.91 (4H), 1.75 (8H), 1.48 (2H), 1.22 (26H), 0.91 (3H). ¹³C NMR (D₂O, ppm): 168.8, 53.4, 48.0, 47.3, 47.1 (m), 45.4, 44.7, 36.7, 32.0, 30.6, 29.9 (m), 29.8, 29.5, 29.4, 29.1, 26.5, 25.9, 25.6, 25.5, 23.8, 22.9, 22.8, 21.7, 13.9. LC/MS (ret time, 7.2 min), calcd for C₃₂H₇₀N₆O m/z 555, obsd 556 (MH⁺).

D-Lys-ε-(3,3-dimethyl-1-butyl)-N¹-spermine (34)—TLC analysis (b); R_{f}=0.06. ¹H NMR (D₂O, δ): 3.91 (1H), 3.33 (1H), 3.23 (1H), 3.05 (14H), 2.01 (2H), 1.86 (4H), 1.71 (4H), 1.67 (2H), 1.50 (2H), 1.38 (2H), 0.85 (9H). LC/MS (ret time, 6.1 min), calcd for C₂₂H₅₀N₆O m/z 415, obsd 416 (MH⁺).

D-Lys-ε-(3-methylpropyl)-N¹-spermine (35)—TLC analysis (b); R_{f}=0.06. ¹H NMR (D₂O, δ): 3.90 (1H), 3.32 (1H), 3.24 (1H), 3.05 (1OH), 2.82 (2H), 2.02 (2H), 1.82 (6H), 1.71 (6H), 1.37 (1H), 0.90 (6H). LC/MS (ret time, 5.8 min), calcd for C₂₀H₄₆N₆O m/z 387, obsd (MH⁺).

L-Lys-ε-(bis-(cyclohexyl))-N¹-spermine (38)—TLC analysis (b); R_{f}=0.22. ¹H NMR (D₂O, δ): 3.96 (1H), 3.30 (2H), 3.04 (18H), 2.06 (2H), 1.86 (4H), 1.72 (16H), 1.40 (2H), 1.18 (6H), 0.97 (4H). ¹³C NMR (D₂O, ppm): 169.8, 60.2, 54.1, 53.2, 47.0 (m), 45.3, 44.6, 36.6 (m), 32.9, 30.3 (m), 25.4, 25.0, 23.8, 22.8, 22.3, 21.7. LC/MS (ret time, 6.4 min), calcd for C₃₀H₆₂N₆O m/z 523, obsd 524 (MH⁺).

D-Lys-ε-(4-phenylbenzyl)-N¹-spermine (39)—TLC analysis (b); R_{f}=0.11. ¹H NMR (D₂O, δ): 7.55 (9H), 4.21 (s, 2H), 3.93 (1H), 3.32 (1H), 3.24 (1H), 3.04 (12H), 2.04 (2H), 1.80 (4H), 1.72 (6H), 1.40 (2H). ¹³C NMR (D₂O, ppm): 169.8, 141.6, 139.6, 130.5, 139.9, 129.3, 128.2, 127.6, 127.0, 53.1, 50.6, 47.1, 47.0, 46.5, 45.3, 44.6, 36.6 (m), 30.3, 25.5, 25.1, 23.8, 22.9 (m), 21.6. LC/MS (ret time, 6.3 min), calcd for C₂₉H₄₈N₆O m/z 497, obsd 498 (MH⁺).

### EXAMPLE 9

### Rapid-throughput Fluorescence Displacement Assay for Quantifying Binding Affinities to LPS

The BODIPY-TR-cadaverine (BC; (5-((4-(4,4-difluoro-5-(2-thienyl)-4-bora-3a,4a-diaza-s-indacene-3-yl) phenoxy)acetyl)amino)pentylamine, hydrochloride); obtained from Molecular probes, Inc., Eugene, Oreg.) displacement assay to quantify the affinities of binding of compounds to LPS has been described in detail recently.⁴⁶ This assay is performed in a rapid-throughput format as follows. The first column (16 wells) of a Corning Nonbinding Surface 384-well flat-bottom black fluorescence microplate contains 15 test compounds plus polymyxin B, all at 5 mM, and are serially two-fold diluted across the remaining 23 columns, achieving a final dilution of 0.596 nM in a volume of 40 μl. Polymyxin B (PMB), a peptide antibiotic known to bind and neutralize LPS⁴⁷ serves as the positive control and reference compound for every plate, enabling the quantitative assessment of repeatability and reproducibility (CV and Z′ factors) for the assay. Robotic liquid handling is performed on a Precision 2000 automated microplate pipetting system, programmed using the Precision Power software, Bio-Tek Instruments Inc., VT, USA. Stock solutions of LPS (5 mg/ml; E. coli 0111:B4; procured from Sigma) and BC (500 μM) are prepared in Tris buffer (pH 7.4, 50 mM). 1 ml each of the LPS and BC stocks are mixed and diluted in Tris buffer to a final volume of 100 ml, yielding final concentrations of 50 μg/ml of LPS and 5 μM BC. 40 μl of this BC:LPS mixture is added to each well of the plate using the Precision 2000. Fluorescence measurements are made at 25° C. on a Fluoromax-3 with Micromax Microwell 384-well plate reader, using DataMax software, Jobin Yvon Inc., N.J. The BC excitation wavelength is 580 nm, emission spectra are taken at 620 nm with both emission and excitation monochromator bandpasses set at 5 nm. The fluorescence of BC is quenched upon binding to LPS, and the displacement of BC by the compounds results in de-quenching (intensity enhancement) of BC fluorescence. Effective displacements (ED₅₀) are computed at the midpoint of the fluorescence signal versus compound concentration displacement curve, determined using an automated four-parameter sigmoidal fit utility of the Origin plotting software (Origin Lab Corp., Mass.), as described in the preceding paper. Z′ factors⁵² computed using the equation: 1-[3(SD+SD′)/(A−A′)] where SD and SD′, A and A′ are standard deviations for the signal and noise, and means of signal and noise, respectively, yielded a Z′ factor of 0.821 and an inter-plate CVs of 5.2% .

### EXAMPLE 10

### Nitric Oxide Assay

Nitric oxide production is measured as total nitrite in murine macrophage J774.A1 cells using the Griess reagent system.^{53;54} Murine macrophage J774.A1 cells are grown in RPMI-1640 cell-culture medium containing L-glutamine and sodium bicarbonate and supplemented with 10% fetal bovine serum, 1% L-glutamine-penicillin-streptomycin solution, and 200 μg/ml L-arginine at 37° C. in a 5% CO₂ atmosphere. Cells are plated at ˜2×10⁶/ml in a volume of 40 μl/well, in 384 well, flat-bottomed, cell culture treated microtiter plates until confluency and subsequently stimulated with 100 ng/ml lipopolysaccharide (LPS). Concurrent to LPS stimulation, serially diluted concentrations of test compounds are added to the cell medium and left to incubate overnight for 16 h. Polymyxin B is used as reference compound in each plate. Positive—(LPS stimulation only) and negative-controls (J774.A1 medium only) are included in each experiment. Nitrite concentrations are measured adding 30 μl of supernatant to equal volumes of Griess reagents (50 μl/well; 0.1% NED solution in ddH₂O and 1% sulfanilamide, 5% phosphoric acid solution in ddH₂O) and incubating for 15 minutes at room temperature in the dark. Absorbance at 535 nm is measured using a Molecular Devices Spectramax M2 multifunction plate reader (Sunnyvale, Calif.). Nitrite concentrations are interpolated from standard curves obtained from serially diluted sodium nitrite standards.

### EXAMPLE 11

### Multiplexed Cytokine Assay ex vivo in Human Blood

100 μl aliquots of fresh whole blood, anticoagulated with EDTA, obtained by venipuncture from healthy human volunteers with informed consent and as per guidelines approved by the Human Subjects Experimentation Committee, is exposed to an equal volume of 50 ng/ml of E. coli 0111:B4 LPS, with graded concentrations of test compounds diluted in saline for 4 h in a 96-well microtiter plate. The effect of the compounds on modulating cytokine production examined using a FACSArray multiplexed flow-cytometric bead array (CBA) system (Becton-Dickinson-Pharmingen, San Jose, Calif.). The system uses a sandwich ELISA-on-a-bead principle,^{55;56} and is comprised of 6 populations of microbeads that are spectrally unique in terms of their intrinsic fluorescence emission intensities (detected in the FL3 channel of a standard flow cytometer). Each bead population is coated with a distinct capture antibody to detect six different cytokines concurrently from biological samples (the human inflammation CBA kit includes TNF-α, IL-1β, IL-6, IL-8, IL-10, and IL-12p70). The beads are incubated with 30 μl of sample, and the cytokines of interest are first captured on the bead. After washing the beads, a mixture of optimally paired second antibodies conjugated to phycoerythrin is added which then forms a fluorescent ternary complex with the immobilized cytokine, the intensity (measured in the FL2 channel) of which is proportional to the cytokine concentration on the bead. The assay is performed according to protocols provided by the vendor. Standard curves are generated using recombinant cytokines provided in the kit. The data are analyzed in the CBA software suite that is integral to the FACSArray system.

### EXAMPLE 12

### Mouse Lethality Experiments

Female, outbred, 9- to 11-week-old CF-1 mice (Charles River, Wilmington, Mass.) weighing 22-28 g are used as described elsewhere.³⁷ Upon arrival, the mice are allowed to acclimatize for a week prior to experimentation, housed 5 per cage in a controlled environment at the AALAC-accredited University of Kansas Animal Care Facility, and allowed access to mouse chow and water ad libitum. The animals are sensitized to the lethal effects of LPS by D-galactosamine.^{55;57;58} The lethal dose causing 100% mortality (LD₁₀₀) dose of the batch of LPS that is used (E. coli 0111:B4 procured from Sigma) is first determined by administering D-galactosamine (800 mg/kg) and LPS (0, 10, 20, 50, 100, 200 ng/mouse) as a single injection intraperitoneally (i.p.) in freshly prepared saline to batches of five animals in a volume of 0.2 ml. The expected dose-response profile is observed in two independent experiments with all five mice receiving 100 ng succumbing within 24 h, establishing the LD₁₀₀ dose to be 100 ng/mouse. In experiments designed to test dose-response effects of the acyl-spermines in affording protection against LPS-induced lethality, mice receive graded doses of compound diluted in saline, i.p., in one flank, immediately before a supralethal (200 ng) LPS challenge, which is administered as a separate i.p. injection into the other flank. In experiments in which the temporal window of protection is to be examined, a fixed dose of 200 μg/mouse of compound is administered at various times, before, or after supralethal (200 ng/mouse) LPS challenge. Lethality is determined at 24 h post LPS challenge.

Compounds according to this disclosure can be combined with pharmaceutically acceptable carriers. The pharmaceutically acceptable carriers include, for example, vehicles, adjuvants, excipients, or diluents, and are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds of this disclosure can be administered by any conventional method available for use in conjunction with pharmaceuticals, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism or ointment. The active ingredient can be administered employing a sustained or delayed release delivery system or an immediate release delivery system.

Formulations suitable for oral administration can contain (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present disclosure, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl B3-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouth washes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to an animal, particularly a human, in the context of the present invention should be sufficient to effect a therapeutic response in the animal over a reasonable time frame. One skilled in the art will recognize that dosage will depend upon a variety of factors including a condition of the animal, the body weight of the animal, as well as the condition being treated.

A suitable dose is that which will result in a concentration of the active agent in a patient which is known to effect the desired response.

The size of the dose also will be determined by the route, timing and frequency of administration as well as the existence, nature, and extent of any adverse side effects that might accompany the administration of the compound and the desired physiological effect.

Useful pharmaceutical dosage forms for administration of the compounds according to the present invention can be illustrated as follows:

Hard Shell Capsules

A large number of unit capsules are prepared by filling standard two-piece hard gelatine capsules each with 100 mg of powdered active ingredient, 150 mg of lactose, 50 mg of cellulose and 6 mg of magnesium stearate.

Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets

A large number of tablets are prepared by conventional procedures so that the dosage unit is 100 mg of active ingredient, 0.2 mg of colloidal silicon dioxide, 5 mg of magnesium stearate, 275 mg of microcrystalline cellulose, 11 mg of starch, and 98.8 mg of lactose. Appropriate aqueous and non-aqueous coatings may be applied to increase palatability, improve elegance and stability or delay absorption.

Immediate Release Tablets/Capsules

These are solid oral dosage forms made by conventional and novel processes. These units are taken orally without water for immediate dissolution and delivery of the medication. The active ingredient is mixed in a liquid containing ingredients such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Moreover, the compounds of the present disclosure can be administered in the form of nose drops, or metered dose and a nasal or buccal inhaler. The drug is delivered from a nasal solution as a fine mist or from a powder as an aerosol.

The foregoing description illustrates and describes the disclosure. Additionally, the disclosure shows and describes only the preferred embodiments but, as mentioned above, it is to be understood that it is capable of use in various other combinations, modifications, and environments and is capable of changes or modifications within the scope of the invention concept as expressed herein, commensurate with the above teachings and/or the skill or knowledge of the relevant art. The embodiments described hereinabove are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended that the appended claims be construed to include alternative embodiments.

All publications and patent applications cited in this specification are herein incorporated by reference, and for any and all purposes, as if each individual publication or patent application were specifically and individually indicated to be incorporated by reference. As used herein, the terms “a”, “an”, and “any” are each intended to include both the singular and plural forms.

### REFERENCE LIST

1. Rietschel, E. T.; Kirikae, T.; Schade, F. U.; Mamat, U.; Schmidt, G.; Loppnow, H.; Ulmer, A. J.; Zahringer, U.; Seydel, U.; Di Padova, F.; and et, a. Bacterial endotoxin: molecular relationships of structure to activity and function. FASEB J. 1994, 8, 217-225.
2. Rietschel, E. T.; Brade, L.; Lindner, B.; and Zahringer, U. Biochemistry of lipopolysaccharides. In Bacterial endotoxic lipopolysaccharides, vol. I. Molecular biochemistry and cellular biology. Morrison, D. C. and Ryan, J. L. Eds.; CRC Press: Boca Raton, 1992; pp 1-41.
3. Hurley, J. C. Antibiotic-induced release of endotoxin: A reappraisal. Clin. Infect. Dis. 1992, 15, 840-854.
4. Hurley, J. C. Antibiotic-induced release of endotoxin. A therapeutic paradox. Drug Saf. 1995, 12, 183-195.
5. Prins, J. M.; van Agtmael, M. A.; Kuijper, E. J.; van Deventer, S. J.; and Speelman, P. Antibiotic-induced endotoxin release in patients with gram-negative urosepsis: a double-blind study comparing imipenem and ceftazidime. J. Infect. Dis. 1995, 172, 886-891.
6. Gelfand, J. A. and Shapiro, L. Cytokines and sepsis: pathophysiology and therapy. New Horizons 1993, 1, 13-22.
7. Gasche, Y.; Pittet, D.; and Sutter, P. Outcome and prognostic factors in bacteremic sepsis. In Clinical trials for treatment of sepsis. Sibbald, W. J. and Vincent, J. L. Eds.; Springer-Verlag: Berlin, 1995; pp 35-51.
8. Centers for Diseases Control. Increases in national hospital discharge survey rates for septicemia—United States, 1979-1987. MMWR 1990, 39, 31-34.
9. Martin, G. S.; Mannino, D. M.; Eaton, S.; and Moss, M. The epidemiology of sepsis in the United States from 1979 through 2000. N. Engl. J. Med. 2003, 348, 1546-1554.
10. Ulevitch, R. J. Molecular mechanisms of innate immunity. Immunol. Res. 2000, 21, 49-54.
11. Ulevitch, R. J. and Tobias, P. Recognition of gram-negative bacteria and endotoxin by the innate immune system. Curr. Opin. Immunol. 1999, 11, 19-23.
12. Dinarello, C. A. Cytokines as mediators in the pathogenesis of septic shock. Curr. Top. Microbiol. Immunol. 1996, 216, 133-165.
13. Dinarello, C. A. The proinflammatory cytokines interleukin-1 and tumor necrosis factor and treatment of the septic shock syndrome. J. Infect. Dis. 1991, 163, 1177-1184.
14. Meyer, J. and Traber, D. L. Nitric oxide and endotoxin shock. Cardiovasc. Res. 1992, 26, 558.
15. Wright, C. E.; Rees, D. D.; and Moncada, S. Protective and pathological roles of nitric oxide in endotoxin shock. Cardiovasc. Res. 1992, 26, 48-57.
16. Bone, R. C.; Balk, R. A.; Cerra, F. B.; Dellinger, R. P.; Fein, A. M.; Knaus, W. A.; Schein, R. M.; and Sibbald, W. J. Definitions for sepsis and organ failure and guidelines for the use of innovative therapies in sepsis. The ACCP/SCCM Consensus Conference Committee. American College of Chest Physicians/Society of Critical Care Medicine. Chest 1992, 101, 1644-1655.
17. Bone, R. C. The sepsis syndrome. Definition and general approach to management. Clin. Chest Med. 1996,17, 175-181.
18. Galanos, C.; Ltideritz, O.; Rietschel, E. T.; Westphal, O.; Brade, H.; Brade, L.; Freudenberg, M. A.; Schade, U. F.; Imoto, M.; Yoshimura, S.; Kusumoto, S.; and Shiba, T. Synthetic and natural Escherichia coli free lipid A express identical endotoxic activities. Eur. J. Biochem. 1985, 148, 1-5.
19. Imoto, M.; Yoshimura, H.; Kusumoto, S.; and Shiba, T. Total synthesis of lipid A, active principle of bacterial endotoxin. Proc. Japan. Acad. Sci. 1984, 60, 285-288.
20. Kotani, S.; Takada, H.; Tsujimoto, M.; Ogawa, T.; Takahashi, I.; Ikeda, T.; Otsuka, K.; Shimauchi, H.; Kasai, N.; Mashimo, J.; Nagao, S.; Tanaka, A.; Tanaka, S.; Harada, K.; Nagaki, K.; Kitamura, H.; Shiba, T.; Kusumoto, S.; Imoto, M.; and Yoshimura, H. Synthetic lipid A with endotoxic and related biological activities comparable to those of a natural lipid A from an Escherichia coli Re-mutant. Infect. Immun. 1985, 49, 225-237.
21. Kusumoto, S.; Kamikawa, T.; Kurosawa, M.; Fukase, K.; Shiba, T.; Kusama, T.; Soga, T.; Shioya, E.; Nakayama, K.; Nakayima, H.; Osada, Y.; and Ono, Y. New results in the synthesis of lipid A and endotoxins. In Endotoxin Research Series, Nowotny, A., Ed., Vol. 1, Cellular and molecular aspects of endotoxin reactions. Nowotny, A., Spitzer, J. J., and Ziegler, E. J. Eds.; Elsevier Science Publishers: Amsterdam, 1990; pp 41-51.
22. Holst, O.; Ulmer, A. J.; Brade, H.; and Rietschel, E. T. On the chemistry and biology of bacterial endotoxic lipopolysaccharides. In Immunotherapy of infections. Masihi, N. Ed.; Marcel Dekker, Inc.: New York, Basel, Hong Kong, 1994; pp 281-308.
23. Rietschel, E. T.; Wollenweber, H. W.; Sidorczyk, Z.; Zahringer, U.; and Luideritz, O. Analysis of the primary structure of lipid A. In Bacterial Lipopolysaccharides: Structure, Synthesis and Biological Activities. Anderson, L. and Unger, F. M. Eds.; Am. Chem. Soc. Symp. Ser.: Washington, D.C., 1983; pp 195-212.
24. Ferguson, A. D.; Hofmann, E.; Coulton, J.; Diedrichs, K.; and Welte, W. Siderophore-mediated iron transport: Crystal structure of FhuA with bound lipopolysaccharide. Science 1998, 2215-2220.
25. Kirikae, T.; Schade, F. U.; Zahringer, U.; Kirikae, F.; Brade, H.; Kusumoto, S.; Kusama, T.; and Rietschel, E. T. The significance of the hydrophilic backbone and the hydrophobic fatty acid regions of Lipid A on macrophage binding and cytokine induction. FEMS Immunol. Med. Microbiol. 1994, 8, 13-26.
26. Kusumoto, S.; Inage, M.; Chaki, H.; Imoto, M.; Shimamoto, T.; and Shiba, T. Chemical synthesis of lipid A for the elucidation of structure-activity relationships. In Bacterial lipopolysaccharides: structure, synthesis, and biological activities. Anderson, L. and Unger, F. M. Eds.; American Chemical Society: 1983; pp 237-254.
27. David, S. A.; Balaram, P.; and Mathan, V. I. Characterization of the interaction of lipid A and lipopolysaccharide with human serum albumin: implications for an endotoxin-carrier function for albumin. J. Endotoxin. Res. 1995, 2, 99-106.
28. David, S. A. The interaction of lipid A and lipopolysaccharide with human serum albumin. In Endotoxins in health and disease. Brade, H., Opal, S. M., Vogel, S. N., and Morrison, D. C. Eds.; Marcel Dekker: New York, 1999; pp 413-422.
29. Bhattacharjya, S.; David, S. A.; Mathan, V. I.; and Balaram, P. Polymyxin B nonapeptide: Conformations in water and in the lipopolysaccharide-bound state determined by two-dimensional NMR and molecular dynamics. Biopolymers 1997, 41, 251-265.
30. David, S. A.; Bhattacharjya, S.; Mathan, V. I.; and Balaram, P. Elucidation of the conformation of free and LPS-bound polymyxin B nonapeptide in water by 2D—NMR and restrained molecular dynamics methods and molecular modeling of polymyxin-lipid A complex. J. Endotoxin. Res. 1994, 1(suppl), A60.
31. David, S. A.; Balaram, P.; and Mathan, V. 1. Interaction of basic amphiphilic polypeptide antimicrobials, gramicidin S, tyrocidin and efrapeptin, with endotoxic lipid A. Med. Microbiol. Lett. 1993, 2, 42-47.
32. David, S. A.; Mathan, V. I.; and Balaram, P. Interaction of melittin with endotoxic lipid A. Biochim. Biophys. Acta 1992, 1123, 269-274.
33. David, S. A.; Awasthi, S. K.; and Balaram, P. The role of polar and facial amphipathic character in determining lipopolysaccharide-binding properties in synthetic cationic peptides. J. Endotoxin Res. 2000, 6, 249-256.
34. David, S. A.; Bechtel, B.; Annaiah, C.; Mathan, V. I.; and Balaram, P. Interaction of cationic amphiphilic drugs with lipid A: Implications for development of endotoxin antagonists. Biochim. Biophys. Acta 1994, 1212, 167-175.
35. David, S. A.; Mathan, V. I.; and Balaram, P. Interactions of linear dicationic molecules with lipid A: Structural requisites for optimal binding affinity. J. Endotoxin. Res. 1995, 2, 325-336.
36. David, S. A.; Awasthi, S. K.; Wiese, A.; Ulmer, A. J.; Lindner, B.; Brandenburg, K.; Seydel, U.; Rietschel, E. T.; Sonesson, A.; and Balaram, P. Characterization of the interactions of a polycationic, amphiphilic, terminally branched oligopeptide with lipid A and lipopolysaccharide from the deep rough mutant of Salmonella minnesota. J. Endotoxin Res. 1996, 3, 369-379.
37. David, S. A.; Silverstein, R.; Amura, C. R.; Kielian, T.; and Morrison, D. C. Lipopolyamines: novel antiendotoxin compounds that reduce mortality in experimental sepsis caused by gram-negative bacteria. Antimicrob. Agents Chemother. 1999, 43, 912-919.
38. David, S. A.; Perez, L.; and Infante, M. R. Sequestration of bacterial lipopolysaccharide by bis(args) gemini compounds. Bioorg. Med. Chem. Lett. 2002, 12, 357-360.
39. David, S. A. Towards a rational development of anti-endotoxin agents: novel approaches to sequestration of bacterial endotoxins with small molecules (Invited Review). J. Molec. Recognition 2001, 14, 370-387.
40. Behr, J. P.; Demeneix, B.; Loeffler, J. P.; and Perez-Mutul, J. Efficient gene transfer into mammalian primary endocrine cells with lipopolyamine-coated DNA. Proc. Natl. Acad. Sci. USA 1989, 86, 6982-6986.
41. Behr, J. P. Gene transfer with synthetic cationic amphiphiles: Prospects for gene therapy. Bioconjug Chem. 1994, 5, 382-389.
42. Felgner, P. L.; Gadek, T. R.; Holm, M.; Roman, R.; Chan, H. W.; Wenz, M.; Northrop, J. P.; Ringold, G. M.; and Danielsen, M. Lipofection: a highly efficient, lipid-mediated DNA transfection procedure. Proc. Natl. Acad. Sci. USA 1987, 84, 7413-7417.
43. San, H.; Yang, Z. Y.; Pompili, V. J.; Jaffe, M. L.; Plautz, G. E.; Xu, L.; Felgner, J.; Wheeler, C. J.; Felgner, P. L.; and Gao, X. Safety and short-term toxicity of a novel cationic lipid formulation for human gene therapy. Hum. Gene Ther. 1993, 4, 781-788.
44. Blagbrough, I. S.; Geall, A. J.; and David, S. A. Lipopolyamines incorportaing the teraamine spermine bound to an alkyl chain, sequester bacterial lipopolysaccharide. Bioorg. Med. Chem. Lett. 2000, 10, 1959-1962.
45. Burns, M. R.; Carlson, C. L.; Vanderwerf, S. M.; Ziemer, J. R.; Weeks, R. S.; Cai, F.; Webb, H. K.; and Graminski, G. F. Amino acid/spermine conjugates: polyamine amides as potent spermidine uptake inhibitors. J. Med. Chem. 2001, 44, 3632-3644.
46. Wood, S. J.; Miller, K. A.; and David, S. A. Anti-endotoxin agents. 1. Development of a fluorescent probe displacement method for the rapid identification of lipopolysaccharide-binding agents. Comb. Chem. High. Throughput. Screen. 2004, 7, 239-249.
47. Morrison, D. C. and Jacobs, D. M. Binding of polymyxin B to the lipid A portion of bacterial lipopolysaccharides. Immunochemistry 1976, 13, 813-818.
48. David, S. A.; Balasubramanian, K. A.; Mathan, V. I.; and Balaram, P. Analysis of the binding of polymyxin B to endotoxic lipid A and core glycolipid using a fluorescent displacement probe. Biochim. Biophys. Acta 1992, 1165, 147-152.
49. Brade, H.; Moll, H.; and Rietschel, E. T. Structural investigation on the inner core region of lipopolysaccharides from Salmonella minnesota rough mutants. Biomed. Environ. Mass Spectrom. 1985, 12, 602-609.
50. Brade, H.; Brade, L.; and Rietschel, E. T. Structure-activity relationships of bacterial lipopolysaccharides (Endotoxins). Current and Future Aspects. Zbl. Bakt. Hyg., I Abt. Orig. 1988,A 268/2, 151-179.
51. Burns, M. R.; Carlson, C. L.; Vanderwerf, S. M.; Ziemer, J. R.; Weeks, R. S.; Cai, F.; Webb, H. K.; and Graminski, G. F. Amino acid/spermine conjugates: polyamine amides as potent spermidine uptake inhibitors. J. Med. Chem. 2001, 44, 3632-3644.
52. Zhang, J. H.; Chung, T. D.; and Oldenburg, K. R. A Simple Statistical Parameter for Use in Evaluation and Validation of High Throughput Screening Assays. J. Biomol. Screen. 1999, 4, 67-73.
53. Green, L. C.; Wagner, D. A.; Glogowski, J.; Skipper, P. L.; Wishnok, J. S.; and Tannenbaum, S. R. Analysis of nitrate, nitrite and [15-N] nitrate in biological fluids. Anal. Biochem. 1982, 126, 131.
54. Greiss, P. Bemerkungen zu der abhandlung der H.H. Weselsky und Benedikt “Ueber einige azoverbindungen”. Chem. Ber. 1879, 12, 426-427.
55. Cook, E. B.; Stahl, J. L.; Lowe, L.; Chen, R.; Morgan, E.; Wilson, J.; Varro, R.; Chan, A.; Graziano, F. M.; and Barney, N. P. Simultaneous measurement of six cytokines in a single sample of human tears using microparticle-based flow cytometry: allergics vs. non-allergics. J. Immunol. Methods 2001, 254, 109-116.
56. Funato, Y.; Baumhover, H.; Grantham-Wright, D.; Wilson, J.; Ernst, D.; and Sepulveda, H. Simultaneous measurement of six human cytokines using the Cytometric Bead Array System, a multiparameter immunoassay system for flow cytometry. Cytometry Res. 2002, 12, 93-103.
57. Freudenberg, M. A. and Galanos, C. Tumor necrosis factor alpha mediates lethal activity of killed Gram-negative abd Gram-positive bacteria in D-galactosamine-treated mice. Infect. Immun. 1991, 59, 2110-2115.

58. Tracey, K. J. and Cerami, A. Tumor necrosis factor, other cytokines and disease. Annu. Rev. Cell Biol. 1993, 9, 317-343.

TABLE 1
Lysine-spermine long acyl chain homologs

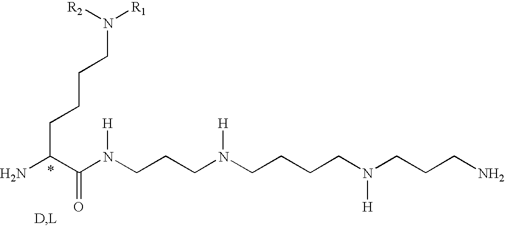

| | | | | | ED₅₀ | NO |
|---|---|---|---|---|---|---|
| ID | R₁ | R₂ | Note | Stereo | (μM) | IC₅₀ (μM) |
| 5 | H | H | | L | 40.42 | >1000 |
| 6 | H | H | | D | 58.42 | >1000 |
| 7 | 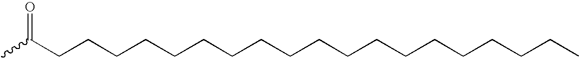 | H | C₂₀ | L | 6.46 | 1.21 |
| 8 | 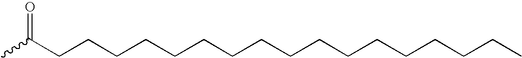 | H | C₁₈ | D | 8.8 | 1.98 |
| 9 | 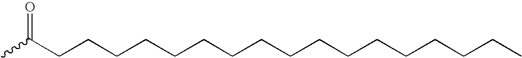 | H | C₁₈ | L | 16.39 | 18.14 |
| 10 | 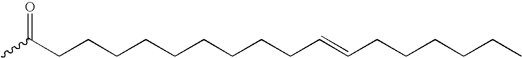 | H | Δ11, C₁₈ | L | 4.2 | NA |
| 11 | 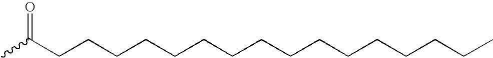 | H | C₁₇ | L | 6.71 | 4.49 |
| 12 | 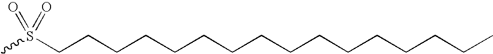 | H | C₁₆ | L | 5.93 | NA |
| 13 | 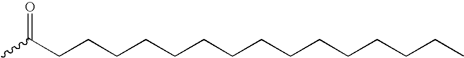 | H | C₁₆ | D | 9.94 | 2.29 |
| 14 | 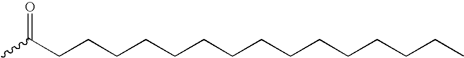 | H | C₁₆ | L | 10.74 | 6.41 |
| 15 | 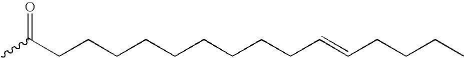 | H | Δ9, C₁₆ | L | 3.82 | 8.85 |
| 16 | 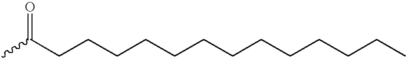 | H | C₁₄ | L | 5.63 | 1.60 |
| 17 | 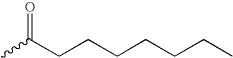 | H | C₈ | L | 12.97 | 162.24 |

TABLE 2
Lysine-spermine mixed acyl analogs

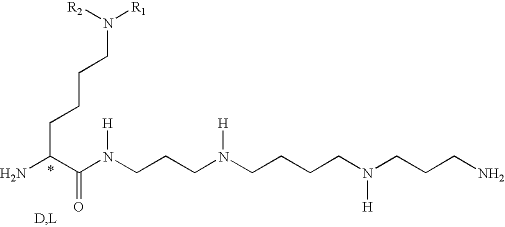

| ID | R₁ | R₂ | Note | Stereo | ED₅₀ (μM) | NO IC₅₀ (μM) |
|---|---|---|---|---|---|---|
| 18 | 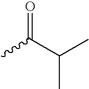 | H | | D | 298.85 | >1000 |
| 19 | 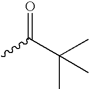 | H | | D | 327.04 | >1000 |
| 20 | 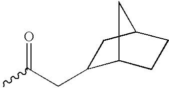 | H | | D | 16.16 | NA |
| 21 | 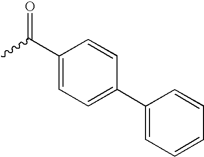 | H | | D | 7.86 | 124.30 |
| 22 | 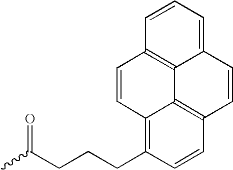 | H | | L | 7.09 | 108.10 |
| 23 | 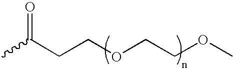 | H | polymer | L | 310.95 | 323.54 |
| 24 | 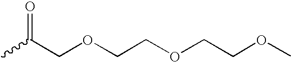 | H | | L | 572.5 | >1000 |
| 25 | 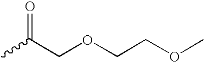 | H | | L | 495.19 | >1000 |

TABLE 3
Lysine-spermine mixed alkyl analogs

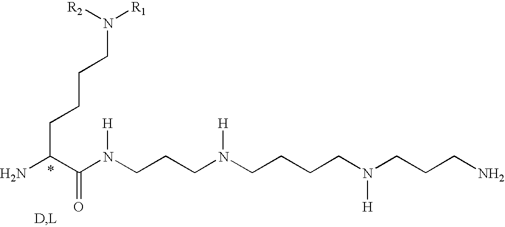

| ID | R₁ | R₂ | Note | Stereo | ED₅₀ (μM) | NO IC₅₀ (μM) |
|---|---|---|---|---|---|---|
| 26 | 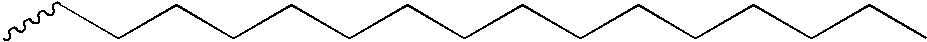 | H | C₁₆ | L | 5.56 | NA |
| 27 | 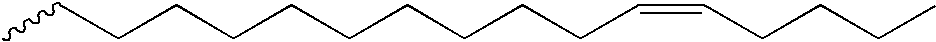 | H | Δ11, C₁₆ | L | 2.59 | 0.66 |
| 28 | 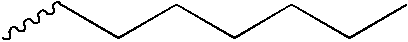 | H | C₇ | D | 3.86 | 82.30 |
| 29 | 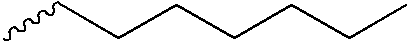 | H | C₇ | L | 5.99 | 46.43 |
| 30 | 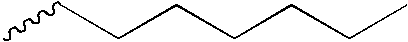 | R₁ | bis, C₇ | L | 2.14 | 38.83 |
| 31 | 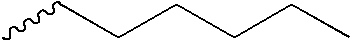 | H | C₆ | D | 7.13 | >1000 |
| 32 | 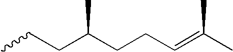 | H | | D | 9.55 | >1000 |
| 33 | 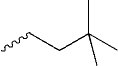 | H | | L | 12.07 | >1000 |
| 34 | 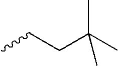 | H | | D | 10.93 | 838.41 |
| 35 | 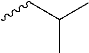 | H | | D | 100.58 | 72.88 |
| 36 | 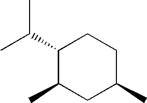 | H | | D | 16.08 | >1000 |
| 37 | 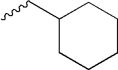 | H | | L | 9.84 | >1000 |
| 38 | 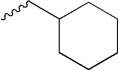 | R₁ | bis | L | 4.04 | >1000 |
| 39 | 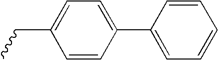 | H | | D | 3.71 | 105.12 |

TABLE 4
Dose-dependent protection of CF-1 mice challenged with a
supralethal dose 200 ng/mouse by compound 8 in cohorts of
five animals. Lethality is recorded at 24 h post-LPS
injection. Ratios denote live/total animals. Asterixes
indicate statistical significance (P < 0.05; Fisher
one-tailed exact test).

| Amount of | No. of live mice/ |
|---|---|
| Compound Used | total no. of mice |
| (μg/mouse) | tested |
| 0 | 0/5 |
| 10 | 0/5 |
| 50 | 1/5 |
| 100 | 4/5* |
| 200 | 5/5* |

TABLE 5
Time-course protection afforded by 8 in the D-galactosamine
sensitized CF-1 mouse lethality model. Animals are injected
with 200 μg of 8 intraperitoneally at times noted with respect
to LPS challenge (200 ng/mouse). Lethality is recorded at 24 h
following LPS injection. Asterixes indicate statistical
significance (P < 0.05; Fisher one-tailed exact test).

| Time of LPS | No. of live mice/total |
|---|---|
| Administration | number of mice tested |
| −6 h | 3/5 |
| −4 h | 4/5* |
| −2 h | 4/5* |
| 0 h | 4/5* |
| +1 h | 0/5* |
| +2 h | 2/5 |

TABLE 6
Time-course protection afforded by compound 8 in the
D-galactosamine sensitized CF-1 mouse lethality model.
Animals are injected with 200 μg of 8 subcutaneously at
times noted with respect to LPS challenge (200 ng/mouse).
Lethality is recorded at 24 h following LPS injection.
Asterixes indicate statistical significance (P < 0.05;
Fisher one-tailed exact test).

| | |
|---|---|
| Time of LPS | No. of live mice/total no. |
| Administration | of mice tested |

| | | |
|---|---|---|
| −24 | h | 2/5 |
| −16 | h | 3/5 |
| −12 | h | 3/5 |
| −8 | h | 3/5 |
| −4 | h | 5/5* |
| 0 | h | 1/5 |
| +2 | h | 1/5 |

TABLE 7
Focused library of acyl and alkyl Lysine-spermine conjugates

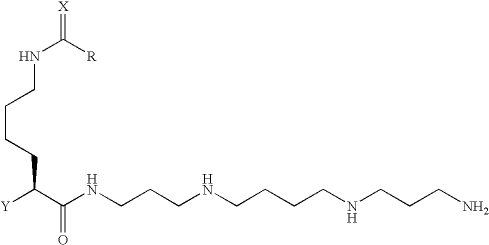

| | | | | | | | | | | MW |
|---|---|---|---|---|---|---|---|---|---|---|
| | R | | | Stereo- | | | NFκ | | MW | (HCl |
| Analog | group | X | Y | chemistry | ED₅₀ | NO | B | (MQT#) | (fb) | salt) |
| 16 | C₁₄ | O | —NH₂ | L | 3.64 | 22.2 | 0.753 | 1546 | 540.88 | 686.72 |
| 14 | C₁₆ | O | —NH₂ | L | 3.05 | 16.8 | 0.819 | 1483 | 568.94 | 714.78 |
| 9 | C₁₈ | O | —NH₂ | L | 3.78 | 8.78 | 0.736 | 1535 | 596.99 | 742.83 |
| 7 | C₂₀ | O | —NH₂ | L | 5.49 | 4.69 | 1.24 | 1531 | 625.05 | 770.89 |
| 41 | C₁₄ | O | —NH₂ | D | 3.22 | 26.0 | 1.21 | 3935 | 540.88 | 686.72 |
| 13 | C₁₆ | O | —NH₂ | D | 3.85 | 13.0 | 0.847 | 1501 | 568.94 | 714.78 |
| 8 | C₁₈ | O | —NH₂ | D | 5.14 | 10.3 | 0.786 | 1576 | 596.99 | 742.83 |
| 42 | C₂₀ | O | —NH₂ | D | 6.75 | 5.77 | 0.126 | 3936 | 625.05 | 770.89 |
| 43 | C₁₄ | H, H | —NH₂ | L | 2.28 | 6.17 | 0.419 | 3937 | 526.89 | 709.19 |
| 26 | C₁₆ | H, H | —NH₂ | L | 3.36 | 3.78 | 0.190 | 1569 | 554.95 | 737.25 |
| 44 | C₁₈ | H, H | —NH₂ | L | 4.38 | 2.25 | 0.337 | 3938 | 583.01 | 765.31 |
| 45 | C₂₀ | H, H | —NH₂ | L | 5.27 | 3.67 | 0.689 | 3939 | 611.07 | 793.37 |
| 46 | C₁₄ | H, H | —NH₂ | D | 2.39 | 5.34 | 0.752 | 3940 | 526.89 | 709.19 |
| 47 | C₁₆ | H, H | —NH₂ | D | 3.26 | 6.89 | 0.350 | 3941 | 554.95 | 737.25 |
| 48 | C₁₈ | H, H | —NH₂ | D | 3.43 | 3.08 | 0.324 | 3942 | 583.01 | 765.31 |
| 49 | C₂₀ | H, H | —NH₂ | D | 3.58 | 2.2 | 1.03 | 3943 | 611.07 | 793.37 |
| 50 | C₁₄ | O | —H | — | 5.88 | 48.4 | 2.67 | 3944 | 525.86 | 635.24 |
| 51 | C₁₆ | O | —H | — | 3.22 | | | 3945 | 553.92 | 663.30 |
| 52 | C₁₈ | O | —H | — | 5.81 | 29.6 | 2.35 | 3946 | 581.98 | 691.36 |
| 53 | C₂₀ | O | —H | — | 124. | 26.6 | 3.88 | 3947 | 610.04 | 719.42 |
| 54 | C₁₄ | H, H | —H | — | 2.57 | 9.48 | 1.48 | 3948 | 511.87 | 657.71 |
| 55 | C₁₆ | H, H | —H | — | 4.84 | 6.12 | 0.952 | 3949 | 539.93 | 685.77 |
| 56 | C₁₈ | H, H | —H | — | 4.28 | 5.34 | 1.10 | 3950 | 567.99 | 713.83 |
| 57 | C₂₀ | H, H | —H | — | 5.12 | 5.02 | 1.49 | 3951 | 596.05 | 741.89 |

What is claimed is:

1. A method for treating endotoxic shock condition or for inhibiting at least one of NO activity, TNF-α production, IL-6 production and cytokine activity by administering to a host in need thereof an effective amount of at least one compound represented by the formula: 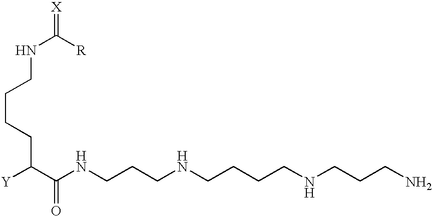
wherein X is O or H, H; R is a hydrophobic C₁₂-C₂₀ chain and Y is —NH₂ or —H; pharmaceutically acceptable salts thereof and prodrugs thereof .

2. The method of claim 1 being for treating endotoxic shock condition.

3. The method of claim 1 being for inhibiting NO activity.

4. The method of claim 1 being for inhibiting TNF-α production.

5. The method of claim 1 being for inhibiting IL-6 production.

6. The method of claim 1 being for inhibiting cytokine activity.

7. The method of claim 1 wherein said hydrophobic C₁₂-C₂₀ chain is an aliphatic chain.

8. The method of claim 1 wherein said hydrophobic C₁₂-C₂₀ chain is an acyl chain.

9. The method of claim 1 wherein said hydrophobic C₁₂-C₂₀ chain contains an SO₂ group in the a position.

10. The method of claim 1 wherein said hydrophobic C₁₂-C₂₀ chain is a phenylbenzyl group.

11. The method of claim 1 wherein said hydrophobic C12-C₂₀ chain is an ethylenically unsaturated aliphatic chain.

12. The method of claim 1 wherein said hydrophobic C]₂-C₂₀ chain is a saturated aliphatic chain.

13. The method of claim 1 wherein X is O.

14. The method of claim 1 wherein said compound is L-Lys-ε-(stearoyl)-N¹-spermine.

15. The method of claim 1 wherein said compound is D-Lys-ε-(stearoyl)-N¹-spermine.

16. The method of claim 1 wherein said compound is L-Lys-ε-(octadecanyl)-N¹-spermine.

17. The method of claim 1 wherein said compound is D-Lys-ε-(octadecanyl)-N¹-spermine.

18. The method of claim 1 involves treatment of sepsis.

19. The method of claim 1 involves treatment of inflammation.

20. The method of claim 1 involves treatment of infections.

21. The method of claim 1 wherein said compound is represented by the formula:

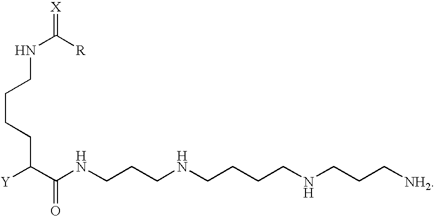

22. A compound represented by the formula 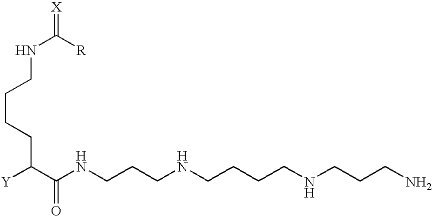
wherein X is O or H, H; R is a hydrophobic C₁₂-C₂₀ chain, pharmaceutically acceptable salts thereof and prodrugs thereof.

23. A pharmaceutical composition comprising at least one compound according to claim 22 and a pharmaceutically acceptable carrier.
